(19) United States
(12) Patent Application Publication
(10) Pub. No.: US 2020/0262925 A1
(43) Pub. Date: Aug. 20, 2020

(54) Combination Therapy For Treatment Of B-Cell Malignancies

(71) Applicant: Janssen Biotech, Inc., Horsham, PA (US)

(72) Inventor: Sriram Balasubramanian, Spring House, PA (US)

(21) Appl. No.: 16/791,217

(22) Filed: Feb. 14, 2020

Related U.S. Application Data
(60) Provisional application No. 62/806,148, filed on Feb. 15, 2019.

(51) Int. Cl. C07K 16/28; A61K 31/519; A61P 35/00

(52) U.S. Cl. CPC C07K 16/2818; A61K 9/0053; A61P 35/00; A61K 31/519

(57) ABSTRACT

Provided herein are methods of treating a B-cell malignancy, and gene mutations that can be used to identify subjects who will be responsive to treatment of a B-cell malignancy with a combination of ibrutinib and an anti-PD-1 antibody.

### CROSS REFERENCE TO RELATED APPLICATIONS

This application claims priority to U.S. Provisional Application No. 62/806,148, filed Feb. 15, 2019, the disclosure of which is hereby incorporated by reference in its entirety.

### TECHNICAL FIELD

Provided herein are methods of treating a B-cell malignancy and gene mutations that can be used to identify subjects who will be responsive to treatment of a B-cell malignancy with a combination of ibrutinib and an anti-PD-1 antibody.

### BACKGROUND

Novel targeted therapies and immuno-oncology agents have revolutionized the treatment of hematologic B-cell malignancies, particularly for difficult-to-treat patients with relapsed/refractory (R/R) diseases. Many patients with follicular lymphoma (FL), diffuse large B-cell lymphoma (DLBCL), and Richter's transformation (RT), however, relapse or become refractory to standard therapies, and the prognosis is poor for those who fail to respond adequately to salvage therapy, or who are ineligible for stem cell transplant. Somatic mutations not only lead to the formation of B-cell malignancies, but can also cause those cancers to become relapsed/refractory. There is a lack of alternative options in heavily-pretreated patients.

### SUMMARY

Disclosed herein are methods of treating a B-cell malignancy in a subject, the method comprising administering to the subject a therapeutically effective amount of a combination of ibrutinib and an anti-PD-1 antibody to thereby treat the B-cell malignancy, wherein:
a) the B-cell malignancy is DLBCL and the subject has one or more mutations in genes selected from KLHL14, RNF213, CSMD3, BCL2, NBPF1, LRP1B, or a combination thereof, wherein the one or more mutations are listed in Table 4 or 6;
b) the B-cell malignancy is GCB-DLBCL and the subject has one or more mutations in genes selected from RNF213, NBPF1, or a combination thereof, wherein the one or more mutations are listed in Table 16;
c) the B-cell malignancy is FL and the subject has one or more mutations in genes selected from BCL2, CREBBP, KMT2D, MUC17, CIITA, FES, NCOA2, TPR, or a combination thereof, wherein the one or more mutations are listed in Table 8 or 10; or
d) the B-cell malignancy is RT and the subject has one or more mutations in genes selected from IRF2BP2, NBPF1, KLHL6, SETX, SF3B1, or a combination thereof, wherein the one or more mutations are listed in Table 12 or 14.

Also provided herein are methods of treating a B-cell malignancy in a subject, the method comprising administering to the subject a therapeutically effective amount of a combination of ibrutinib and an anti-PD-1 antibody to thereby treat the B-cell malignancy, wherein:
a) the B-cell malignancy is DLBCL and the subject does not have one or more mutations in genes selected from TP53, EBF1, ADAMTS20, AKAP9, SOCS1, TNFRSF14, MYD88, NFKB1B, or a combination thereof, wherein the one or more mutations are listed in Table 4 or 6;
b) the B-cell malignancy is GCB-DLBCL and the subject does not have one or more mutations in genes selected from KMT2D, BCL2, CSMD3, CREBBP, EBF1, SGK1, or a combination thereof, wherein the one or more mutations are listed in Table 16;
c) the B-cell malignancy is FL and the subject does not have one or more mutations in genes selected from CREBBP, KMT2D, BCL2, STAT6, NBPF1, EZH2, or a combination thereof, wherein the one or more mutations are listed in Table 8 or 10; or
d) the B-cell malignancy is RT and the subject does not have one or more mutations in genes selected from ROS1, IGLL5, PASK, or a combination thereof, wherein the one or more mutations are listed in Table 12 or 14.

Further provided are methods of predicting a likelihood of responsiveness to a combination of ibrutinib and an anti-PD-1 antibody in a subject having a B-cell malignancy, wherein:
a) the B-cell malignancy is DLBCL and the method comprises analyzing a sample from the subject for one or more mutations in genes selected from KLHL14, RNF213, CSMD3, BCL2, NBPF1, LRP1B, or a combination thereof, wherein the one or more mutations are listed in Table 4 or 6;
b) the B-cell malignancy is GCB-DLBCL and the method comprises analyzing a sample from the subject for one or more mutations in genes selected from RNF213, NBPF1, or a combination thereof, wherein the one or more mutations are listed in Table 16;
c) the B-cell malignancy is FL and the method comprises analyzing a sample from the subject for one or more mutations in genes selected from BCL2, CREBBP, KMT2D, MUC17, CIITA, FES, NCOA2, TPR, or a combination thereof, wherein the one or more mutations are listed in Table 8 or 10; or
d) the B-cell malignancy is RT and the method comprises analyzing a sample from the subject for one or more mutations in genes selected from IRF2BP2, NBPF1, KLHL6, SETX, SF3B1, or a combination thereof, wherein the one or more mutations are listed in Table 12 or 14;

wherein the one or more mutations in the genes are indicative of responsiveness to the combination.

Also disclosed are methods of predicting a likelihood of nonresponsiveness to a combination of ibrutinib and an anti-PD-1 antibody in a subject having a B-cell malignancy, wherein:
a) the B-cell malignancy is DLBCL and the method comprises analyzing a sample from the subject for one or more mutations in genes selected from TP53, EBF1, ADAMTS20, AKAP9, SOCS1, TNFRSF14, MYD88, NFKB1B, or a combination thereof, wherein the one or more mutations are listed in Table 4 or 6;
b) the B-cell malignancy is GCB-DLBCL and the method comprises analyzing a sample from the subject for one or more mutations in genes selected from KMT2D, BCL2, CSMD3, CREBBP, EBF1, SGK1, or a combination thereof, wherein the one or more mutations are listed in Table 16;
c) the B-cell malignancy is FL and the method comprises analyzing a sample from the subject for one or more mutations in genes selected from CREBBP, KMT2D, BCL2, STAT6, NBPF1, EZH2, or a combination thereof, wherein the one or more mutations are listed in Table 8 or 10; or
d) the B-cell malignancy is RT and the method comprises analyzing a sample from the subject for one or more mutations in genes selected from ROS1, IGLL5, PASK, or a combination thereof, wherein the one or more mutations are listed in Table 12 or 14;

wherein the one or more mutations in the genes is indicative of nonresponsiveness to the combination.

### BRIEF DESCRIPTION OF THE DRAWINGS

The summary, as well as the following detailed description, is further understood when read in conjunction with the appended drawings. For the purpose of illustrating the disclosed methods, there are shown in the drawings exemplary embodiments of the methods; however, the methods are not limited to the specific embodiments disclosed. In the drawings:

FIG. 1 illustrates the dosing schedule of the LYM1002 study disclosed herein.

FIG. 2 illustrates a plot of the progression free survival (PFS) by IHC-based PD-L1 expression in DLBCL patients (N=26).

FIG. 3 illustrates a plot of the progression free survival (PFS) by IHC-based PD-L1 expression in germinal center B-cell (GCB) DLBCL patients (N=17).

FIG. 4A, FIG. 4B, FIG. 4C, FIG. 4D, and FIG. 4E illustrate percent progression free survival (PFS) over time in DLBCL and Richter Syndrome subjects. FIG. 4A: PFS in DLBCL subjects with TP53 mutated (TP53 M) vs TP53 wild type (TP53 WT) (p=0.002); FIG. 4B: PFS in DLBCL subjects following 2 courses of ibrutinib plus nivolumab (molecular remission, MR+) vs no molecular remission (MR−); FIG. 4C: PFS in relapsed/refractory DLBCL subjects with TP53 WT MR+, TP53 WT MR−, TP53 M MR+, and TP53 M MR−; FIG. 4D: PFS in Richter Syndrome subjects with TP53 WT vs TP53 M; and FIG. 4E: PFS in Richter Syndrome subjects with MR+vs. MR−.

### DETAILED DESCRIPTION OF ILLUSTRATIVE EMBODIMENTS

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any subcombination.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “comprising” is intended to include examples encompassed by the terms “consisting essentially of” and “consisting of”; similarly, the term “consisting essentially of” is intended to include examples encompassed by the term “consisting of.”

Ibrutinib, a first-in-class, oral, covalent inhibitor of Bruton's tyrosine kinase (BTK), approved for several B-cell malignancies in the United States and other countries, disrupts signaling pathways essential for the adhesion, proliferation, homing, and survival of malignant B cells.

“Treat,” “treatment,” and like terms refer to both therapeutic treatment and prophylactic or preventative measures, and includes reducing the severity and/or frequency of symptoms, eliminating symptoms and/or the underlying cause of the symptoms, reducing the frequency or likelihood of symptoms and/or their underlying cause, and improving or remediating damage caused, directly or indirectly, by the B-cell malignancy. Treatment includes complete response and partial response to the combination (ibrutinib and an anti-PD-1 antibody). Treatment also includes prolonging survival as compared to the expected survival of a subject not receiving treatment. Subjects to be treated include those that have the condition or disorder as well as those prone to have the condition or disorder or those in which the condition or disorder is to be prevented.

As used herein, the phrase “therapeutically effective amount” refers to an amount of the combination of ibrutinib and an anti-PD-1 antibody, as described herein, effective to achieve a particular biological or therapeutic result such as, but not limited to, biological or therapeutic results disclosed, described, or exemplified herein. The therapeutically effective amount may vary according to factors such as the disease state, age, sex, and weight of the individual, and the ability of the composition to cause a desired response in a subject. Exemplary indicators of a therapeutically effect amount include, for example, improved well-being of the patient, reduction of a tumor burden, arrested or slowed growth of the B-cell malignancy, and/or absence of metastasis of the B-cell malignancy cells to other locations in the body.

The term “subject” as used herein is intended to mean any animal, in particular, mammals. Thus, the disclosed methods are applicable to human and nonhuman animals, although most preferably with humans. “Subject” and “patient” are used interchangeably herein.

As used herein, “combination of ibrutinib and an anti-PD-1 antibody” refers to a treatment regimen in which the ibrutinib and the anti-PD-1 antibody are administered substantially at the same time, concurrently, or sequentially. Thus, the ibrutinib and the anti-PD-1 antibody can be comprised in separate compositions to be administered to the subject.

The following abbreviations are used herein: relapsed or refractory (R/R); overall response rate (ORR); overall survival (OS); progression-free survival (PFS); follicular lymphoma (FL); diffuse large B-cell lymphoma (DLBCL); Richter's transformation (RT); chronic lymphocytic leukemia/small lymphocytic lymphoma (CLL/SLL); gene expression profiling (GEP); complete response (CR); partial response (PR); activated B-cell (ABC); germinal center B-cell (GCB); partial response with lymphocytosis (PR-L); progressive disease (PD); and stable disease (SD).

Methods of treating a B-cell malignancy

Provided herein are methods of treating a B-cell malignancy in a subject, wherein the B-cell malignancy is diffuse large B-cell lymphoma (DLBCL), follicular lymphoma (FL), or Richter's transformation (RT). The methods comprise administering to the subject a therapeutically effective amount of a combination of ibrutinib and an anti-PD-1 antibody to thereby treat the B-cell malignancy, wherein the subject has one or more mutations in genes selected from KLHL14, RNF213, CSMD3, BCL2, NBPF1, LRP1B, CREBBP, KMT2D, MUC17, CIITA, FES, NCOA2, TPR, IRF2BP2, KLHL6, SETX, SF3B1, or a combination thereof. In some embodiments, the methods comprise administering to the subject a therapeutically effective amount of a combination of ibrutinib and an anti-PD-1 antibody to thereby treat the B-cell malignancy, wherein:
a) the B-cell malignancy is DLBCL and the subject has one or more mutations in genes selected from KLHL14, RNF213, CSMD3, BCL2, NBPF1, LRP1B, or a combination thereof, wherein the one or more mutations are listed in Table 4 or 6;
b) the B-cell malignancy is GCB-DLBCL and the subject has one or more mutations in genes selected from RNF213, NBPF1, or a combination thereof, wherein the one or more mutations are listed in Table 16;
c) the B-cell malignancy is FL and the subject has one or more mutations in genes selected from BCL2, CREBBP, KMT2D, MUC17, CIITA, FES, NCOA2, TPR, or a combination thereof, wherein the one or more mutations are listed in Table 8 or 10; or
d) the B-cell malignancy is RT and the subject has one or more mutations in genes selected from IRF2BP2, NBPF1, KLHL6, SETX, SF3B1, or a combination thereof, wherein the one or more mutations are listed in Table 12 or 14.

Also provided herein are methods of treating a B-cell malignancy in a subject having one or more mutations in genes selected from KLHL14, RNF213, CSMD3, BCL2, NBPF1, LRP1B, CREBBP, KMT2D, MUC17, CIITA, FES, NCOA2, TPR, IRF2BP2, KLHL6, SETX, SF3B1, or a combination thereof, the methods comprising administering to the subject a therapeutically effective amount of a combination of ibrutinib and an anti-PD-1 antibody to thereby treat the B-cell malignancy wherein:
a) the B-cell malignancy is DLBCL and the subject has one or more mutations in genes selected from KLHL14, RNF213, CSMD3, BCL2, NBPF1, LRP1B, or a combination thereof, wherein the one or more mutations are listed in Table 4 or 6;
b) the B-cell malignancy is GCB-DLBCL and the subject has one or more mutations in genes selected from RNF213, NBPF1, or a combination thereof, wherein the one or more mutations are listed in Table 16;
c) the B-cell malignancy is FL and the subject has one or more mutations in genes selected from BCL2, CREBBP, KMT2D, MUC17, CIITA, FES, NCOA2, TPR, or a combination thereof, wherein the one or more mutations are listed in Table 8 or 10; or
d) the B-cell malignancy is RT and the subject has one or more mutations in genes selected from IRF2BP2, NBPF1, KLHL6, SETX, SF3B1, or a combination thereof, wherein the one or more mutations are listed in Table 12 or 14.

In some embodiments, the B-cell malignancy is DLBCL and the subject has one or more mutations in genes selected from KLHL14, RNF213, CSMD3, BCL2, NBPF1, LRP1B, or a combination thereof, wherein the one or more mutations are listed in Table 4 or 6. In some aspects, the subject has one or more mutations in KLHL14, RNF213, or a combination thereof, wherein the one or more mutations are listed in Table 4 or 6. The methods can be performed on subjects having one or more mutations listed in Table 4 or 6 in 1, 2, 3, 4, 5, or all 6 of KLHL14, RNF213, CSMD3, BCL2, NBPF1, and LRP1B and various combinations thereof.

In some embodiments, the B-cell malignancy is GCB-DLBCL and the subject has one or more mutations in genes selected from RNF213, NBPF1, or a combination thereof, wherein the one or more mutations are listed in Table 16. The methods can be performed on subjects having one or more mutations listed in Table 16 in either or both of RNF213 and NBPF1.

In some embodiments, the B-cell malignancy is FL and the subject has one or more mutations in genes selected from BCL2, CREBBP, KMT2D, MUC17, CIITA, FES, NCOA2, TPR, or a combination thereof, wherein the one or more mutations are listed in Table 8 or 10. In some aspects, the subject has one or more mutations in BCL2, wherein the one or more mutations are listed in Table 8 or 10. The methods can be performed on subjects having one or more mutations listed in Table 8 or 10 in 1, 2, 3, 4, 5, 6, 7, or all 8 of BCL2, CREBBP, KMT2D, MUC17, CIITA, FES, NCOA2, or TPR and various combinations thereof.

In some embodiments, the B-cell malignancy is RT and the subject has one or more mutations in genes selected from IRF2BP2, NBPF1, KLHL6, SETX, SF3B1, or a combination thereof, wherein the one or more mutations are listed in Table 12 or 14. The methods can be performed on subjects having one or more mutations listed in Table 12 or 14 in 1, 2, 3, 4, or all 5 of IRF2BP2, NBPF1, KLHL6, SETX, or SF3B1 and various combinations thereof.

Also disclosed are methods of treating a B-cell malignancy in a subject, the methods comprising administering to the subject a therapeutically effective amount of a combination of ibrutinib and an anti-PD-1 antibody to thereby treat the B-cell malignancy, wherein the subject does not have one or more mutations in genes selected from TP53, EBF1, ADAMTS20, AKAP9, SOCS1, TNFRSF14, MYD88, NFKB1B, KMT2D, BCL2, CSMD3, CREBBP, SGK1, STAT6, NBPF1, EZH2, ROS1, IGLL5, PASK, or a combination thereof. In some embodiments the methods comprise administering to the subject a therapeutically effective amount of a combination of ibrutinib and an anti-PD-1 antibody to thereby treat the B-cell malignancy, wherein:
a) the B-cell malignancy is DLBCL and the subject does not have one or more mutations in genes selected from TP53, EBF1, ADAMTS20, AKAP9, SOCS1, TNFRSF14, MYD88, NFKB1B, or a combination thereof, wherein the one or more mutations are listed in Table 4 or 6;
b) the B-cell malignancy is GCB-DLBCL and the subject does not have one or more mutations in genes selected from KMT2D, BCL2, CSMD3, CREBBP, EBF1, SGK1, or a combination thereof, wherein the one or more mutations are listed in Table 16;
c) the B-cell malignancy is FL and the subject does not have one or more mutations in genes selected from CREBBP, KMT2D, BCL2, STAT6, NBPF1, EZH2, or a combination thereof, wherein the one or more mutations are listed in Table 8 or 10; or
d) the B-cell malignancy is RT and the subject does not have one or more mutations in genes selected from ROS1, IGLL5, PASK, or a combination thereof, wherein the one or more mutations are listed in Table 12 or 14.

Disclosed are methods of treating a B-cell malignancy in a subject not having one or more mutations in genes selected from TP53, EBF1, ADAMTS20, AKAP9, SOCS1, TNFRSF14, MYD88, NFKB1B, KMT2D, BCL2, CSMD3, CREBBP, SGK1, STAT6, NBPF1, EZH2, ROS1, IGLL5, PASK, or a combination thereof, the methods comprising administering to the subject a therapeutically effective amount of a combination of ibrutinib and an anti-PD-1 antibody to thereby treat the B-cell malignancy wherein:
a) the B-cell malignancy is DLBCL and the subject does not have one or more mutations in genes selected from TP53, EBF1, ADAMTS20, AKAP9, SOCS1, TNFRSF14, MYD88, NFKB1B, or a combination thereof, wherein the one or more mutations are listed in Table 4 or 6;
b) the B-cell malignancy is GCB-DLBCL and the subject does not have one or more mutations in genes selected from KMT2D, BCL2, CSMD3, CREBBP, EBF1, SGK1, or a combination thereof, wherein the one or more mutations are listed in Table 16;
c) the B-cell malignancy is FL and the subject does not have one or more mutations in genes selected from CREBBP, KMT2D, BCL2, STAT6, NBPF1, EZH2, or a combination thereof, wherein the one or more mutations are listed in Table 8 or 10; or
d) the B-cell malignancy is RT and the subject does not have one or more mutations in genes selected from ROS1, IGLL5, PASK, or a combination thereof, wherein the one or more mutations are listed in Table 12 or 14.

In some embodiments, the B-cell malignancy is DLBCL and the subject does not have one or more mutations in genes selected from TP53, EBF1, ADAMTS20, AKAP9, SOCS1, TNFRSF14, MYD88, NFKB1B, or a combination thereof, wherein the one or more mutations are listed in Table 4 or 6. The methods can be performed on subjects not having one or more mutations listed in Table 4 or 6 in 1, 2, 3, 4, 5, 6, 7, or all 8 of TP53, EBF1, ADAMTS20, AKAP9, SOCS1, TNFRSF14, MYD88, or NFKB1B and various combinations thereof.

In some embodiments, the B-cell malignancy is GCB-DLBCL and the subject does not have one or more mutations in genes selected from KMT2D, BCL2, CSMD3, CREBBP, EBF1, SGK1, or a combination thereof, wherein the one or more mutations are listed in Table 16. The methods can be performed on subjects not having one or more mutations listed in Table 16 in 1, 2, 3, 4, 5, or all 6 of KMT2D, BCL2, CSMD3, CREBBP, EBF1, or SGK1 and various combinations thereof.

In some embodiments, the B-cell malignancy is FL and the subject does not have one or more mutations in genes selected from CREBBP, KMT2D, BCL2, STAT6, NBPF1, EZH2, or a combination thereof, wherein the one or more mutations are listed in Table 8 or 10. The methods can be performed on subjects not having one or more mutations listed in Table 8 or 10 in 1, 2, 3, 4, 5, or all 6 of CREBBP, KMT2D, BCL2, STAT6, NBPF1, or EZH2 and various combinations thereof.

In some embodiments, the B-cell malignancy is RT and the subject does not have one or more mutations in genes selected from ROS1, IGLL5, PASK, or a combination thereof, wherein the one or more mutations are listed in Table 12 or 14. In some aspects, the subject does not have one or more mutations in ROS1, wherein the one or more mutations are listed in Table 12 or 14. The methods can be performed on subjects not having one or more mutations listed in Table 12 or 14 in 1, 2, or all 3 of ROS1, IGLL5, or PASK and various combinations thereof.

The methods can further comprise, prior to the treating, analyzing a sample from the subject for the presence or absence of the one or more mutations listed in Tables 4, 6, 8, 10, 12, 14, or 16. The methods can also comprise, prior to the analyzing and treating, isolating a sample from the subject. In some embodiments, for example, the methods comprise: isolating a sample from a subject, analyzing the sample from the subject for the presence or absence of the one or more mutations listed in Tables 4, 6, 8, 10, 12, 14, or 16, and treating the subject.

Suitable samples from the subject include, for example, blood or tumor samples. In some aspects, the methods can comprise, prior to the treating, isolating and/or analyzing a blood sample from the subject for the presence or absence of the one or more mutations listed in Tables 4, 6, 8, 10, 12, 14, or 16. In some aspects, the methods can comprise, prior to the treating, isolating and/or analyzing a tumor sample from the subject for the presence or absence of the one or more mutations listed in Tables 4, 6, 8, 10, 12, 14, or 16.

In some embodiments, the anti-PD-1 antibody comprises nivolumab (brand name OPDIVO®).

Suitable amounts of ibrutinib for use in the disclosed methods include from about 140 mg to about 840 mg. In some embodiments, the amount of ibrutinib comprises 140 mg, 190 mg, 240 mg, 290 mg, 340 mg, 390 mg, 420 mg, 440 mg, 490 mg, 540 mg, 590 mg, 640 mg, 690 mg, 740 mg, 790 mg, or 840 mg.

Suitable amounts of the anti-PD-1 antibody include from about 1 mg/kg to about 5 mg/kg. In some embodiments, the amount of the anti-PD-1 antibody comprises 1 mg/kg, 1.5 mg/kg, 2 mg/kg, 2.5 mg/kg, 3 mg/kg, 3.5 mg/kg, 4 mg/kg, 4.5 mg/kg, or 5 mg/kg. In some aspects, the therapeutically effective amount of the combination of ibrutinib and the anti-PD-1 antibody comprises 560 mg of the ibrutinib and 3 mg/kg of the anti-PD-1 antibody.

The anti-PD-1 antibody can be administered intravenously and the ibrutinib can be administered orally. An exemplary dosing schedule includes, for example, the anti-PD-1 antibody administered on a 14-day cycle and the ibrutinib administered once daily.

In some embodiments, the treating results in a complete response (CR) or partial response (PR) in the subject.

Suitable subjects for treatment with the disclosed methods include those with:
a) DLBCL, FL, or RT (transformation from CLL/SLL only);
b) ≥1 prior therapy (≥2 prior therapies for FL) but no more than 4 prior lines of treatment;
c) an Eastern Cooperative Oncology Group (ECOG) performance status ≤2;
d) measurable disease; and
e) no prior ibrutinib or anti-PD-1 therapies.

Also provided herein is a combination of ibrutinib and an anti-PD-1 antibody for use in treating a B-cell malignancy in a subject, wherein:
a) the B-cell malignancy is DLBCL and the subject has one or more mutations in genes selected from KLHL14, RNF213, CSMD3, BCL2, NBPF1, LRP1B, or a combination thereof, wherein the one or more mutations are listed in Table 4 or 6;
b) the B-cell malignancy is GCB-DLBCL and the subject has one or more mutations in genes selected from RNF213, NBPF1, or a combination thereof, wherein the one or more mutations are listed in Table 16;
c) the B-cell malignancy is FL and the subject has one or more mutations in genes selected from BCL2, CREBBP, KMT2D, MUC17, CIITA, FES, NCOA2, TPR, or a combination thereof, wherein the one or more mutations are listed in Table 8 or 10; or
d) the B-cell malignancy is RT and the subject has one or more mutations in genes selected from IRF2BP2, NBPF1, KLHL6, SETX, SF3B1, or a combination thereof, wherein the one or more mutations are listed in Table 12 or 14.

Also provided is the use of ibrutinib in the manufacture of a medicament for, in combination with an anti-PD-1 antibody, treating a B-cell malignancy in a subject, wherein:
a) the B-cell malignancy is DLBCL and the subject has one or more mutations in genes selected from KLHL14, RNF213, CSMD3, BCL2, NBPF1, LRP1B, or a combination thereof, wherein the one or more mutations are listed in Table 4 or 6;
b) the B-cell malignancy is GCB-DLBCL and the subject has one or more mutations in genes selected from RNF213, NBPF1, or a combination thereof, wherein the one or more mutations are listed in Table 16;
c) the B-cell malignancy is FL and the subject has one or more mutations in genes selected from BCL2, CREBBP, KMT2D, MUC17, CIITA, FES, NCOA2, TPR, or a combination thereof, wherein the one or more mutations are listed in Table 8 or 10; or
d) the B-cell malignancy is RT and the subject has one or more mutations in genes selected from IRF2BP2, NBPF1, KLHL6, SETX, SF3B1, or a combination thereof, wherein the one or more mutations are listed in Table 12 or 14.

Disclosed is a combination of ibrutinib and an anti-PD-1 antibody for use in treating a B-cell malignancy in a subject, wherein:
a) the B-cell malignancy is DLBCL and the subject does not have one or more mutations in genes selected from TP53, EBF1, ADAMTS20, AKAP9, SOCS1, TNFRSF14, MYD88, NFKB1B, or a combination thereof, wherein the one or more mutations are listed in Table 4 or 6;
b) the B-cell malignancy is GCB-DLBCL and the subject does not have one or more mutations in genes selected from KMT2D, BCL2, CSMD3, CREBBP, EBF1, SGK1, or a combination thereof, wherein the one or more mutations are listed in Table 16;
c) the B-cell malignancy is FL and the subject does not have one or more mutations in genes selected from CREBBP, KMT2D, BCL2, STAT6, NBPF1, EZH2, or a combination thereof, wherein the one or more mutations are listed in Table 8 or 10; or
d) the B-cell malignancy is RT and the subject does not have one or more mutations in genes selected from ROS1, IGLL5, PASK, or a combination thereof, wherein the one or more mutations are listed in Table 12 or 14.

Also disclosed is use of ibrutinib in the manufacture of a medicament for, in combination with an anti-PD-1 antibody, treating a B-cell malignancy in a subject, wherein:
a) the B-cell malignancy is DLBCL and the subject does not have one or more mutations in genes selected from TP53, EBF1, ADAMTS20, AKAP9, SOCS1, TNFRSF14, MYD88, NFKB1B, or a combination thereof, wherein the one or more mutations are listed in Table 4 or 6;
b) the B-cell malignancy is GCB-DLBCL and the subject does not have one or more mutations in genes selected from KMT2D, BCL2, CSMD3, CREBBP, EBF1, SGK1, or a combination thereof, wherein the one or more mutations are listed in Table 16;
c) the B-cell malignancy is FL and the subject does not have one or more mutations in genes selected from CREBBP, KMT2D, BCL2, STAT6, NBPF1, EZH2, or a combination thereof, wherein the one or more mutations are listed in Table 8 or 10; or
d) the B-cell malignancy is RT and the subject does not have one or more mutations in genes selected from ROS1, IGLL5, PASK, or a combination thereof, wherein the one or more mutations are listed in Table 12 or 14.

### Methods of Predicting a Likelihood of Responsiveness or Nonresponsiveness to a Combination of Ibrutinib and an Anti-PD-1 Antibody in a Subject Having a B-Cell Malignancy

Also provided are methods of predicting a likelihood of responsiveness to a combination of ibrutinib and an anti-PD-1 antibody in a subject having a B-cell malignancy, the method comprising analyzing a sample from the subject for one or more mutations in genes selected from KLHL14, RNF213, CSMD3, BCL2, NBPF1, LRP1B, CREBBP, KMT2D, MUC17, CIITA, FES, NCOA2, TPR, IRF2BP2, KLHL6, SETX, or SF3B1, or a combination thereof, wherein a mutation in the one or more genes is indicative of responsiveness to the combination. In some embodiments:
a) the B-cell malignancy is DLBCL and the method comprises analyzing a sample from the subject for one or more mutations in genes selected from KLHL14, RNF213, CSMD3, BCL2, NBPF1, LRP1B, or a combination thereof, wherein the one or more mutations are listed in Table 4 or 6;
b) the B-cell malignancy is GCB-DLBCL and the method comprises analyzing a sample from the subject for one or more mutations in genes selected from RNF213, NBPF1, or a combination thereof, wherein the one or more mutations are listed in Table 16;
c) the B-cell malignancy is FL and the method comprises analyzing a sample from the subject for one or more mutations in genes selected from BCL2, CREBBP, KMT2D, MUC17, CIITA, FES, NCOA2, TPR, or a combination thereof, wherein the one or more mutations are listed in Table 8 or 10; or
d) the B-cell malignancy is RT and the method comprises analyzing a sample from the subject for one or more mutations in genes selected from IRF2BP2, NBPF1, KLHL6, SETX, SF3B1, or a combination thereof, wherein the one or more mutations are listed in Table 12 or 14;

wherein the one or more mutations in the genes are indicative of responsiveness to the combination.

In some embodiments, the B-cell malignancy is DLBCL and the method comprises analyzing a sample from the subject for one or more mutations in genes selected from KLHL14, RNF213, CSMD3, BCL2, NBPF1, LRP1B, or a combination thereof, wherein the one or more mutations are listed in Table 4 or 6 and the one or more mutations in the genes are indicative of responsiveness to the combination. In some aspects, the B-cell malignancy is DLBCL and the method comprises analyzing a sample from the subject for one or more mutations in genes selected from KLHL14, RNF213, or a combination thereof, wherein the one or more mutations are listed in Table 4 or 6 and the one or more mutations in the genes are indicative of responsiveness to the combination. One or more mutations listed in Table 4 or 6 in 1, 2, 3, 4, 5, or all 6 of KLHL14, RNF213, CSMD3, BCL2, NBPF1, and LRP1B and various combinations thereof can be indicative of responsiveness to the combination.

In some embodiments, the B-cell malignancy is GCB-DLBCL and the method comprises analyzing a sample from the subject for one or more mutations in genes selected from RNF213, NBPF1, or a combination thereof, wherein the one or more mutations are listed in Table 16 and the one or more mutations in the genes are indicative of responsiveness to the combination. One or more mutations as listed in Table 16 in either or both of RNF213 and NBPF1 can be indicative of responsiveness to the combination.

In some embodiments, the B-cell malignancy is FL and the method comprises analyzing a sample from the subject for one or more mutations in genes selected from BCL2, CREBBP, KMT2D, MUC17, CIITA, FES, NCOA2, TPR, or a combination thereof, wherein the one or more mutations are listed in Table 8 or 10 and the one or more mutations in the genes are indicative of responsiveness to the combination. In some aspects, the method comprises analyzing a sample from the subject for one or more mutations in BCL2, wherein the one or more mutations are listed in Table 8 or 10. One or more mutations as listed in Table 8 or 10 in 1, 2, 3, 4, 5, 6, 7, or all 8 of BCL2, CREBBP, KMT2D, MUC17, CIITA, FES, NCOA2, or TPR and various combinations thereof can be indicative of responsiveness to the combination.

In some embodiments, the B-cell malignancy is RT and the method comprises analyzing a sample from the subject for one or more mutations in genes selected from IRF2BP2, NBPF1, KLHL6, SETX, SF3B1, or a combination thereof, wherein the one or more mutations are listed in Table 12 or 14. One or more mutations in 1, 2, 3, 4, or all 5 of IRF2BP2, NBPF1, KLHL6, SETX, or SF3B1 and various combinations thereof can be indicative of responsiveness to the combination.

Methods of predicting a likelihood of nonresponsiveness to a combination of ibrutinib and an anti-PD-1 antibody in a subject having a B-cell malignancy are also provided, wherein:
a) the B-cell malignancy is DLBCL and the method comprises analyzing a sample from the subject for one or more mutations in genes selected from TP53, EBF1, ADAMTS20, AKAP9, SOCS1, TNFRSF14, MYD88, NFKB1B, or a combination thereof, wherein the one or more mutations are listed in Table 4 or 6;
b) the B-cell malignancy is GCB-DLBCL and the method comprises analyzing a sample from the subject for one or more mutations in genes selected from KMT2D, BCL2, CSMD3, CREBBP, EBF1, SGK1, or a combination thereof, wherein the one or more mutations are listed in Table 16;
c) the B-cell malignancy is FL and the method comprises analyzing a sample from the subject for one or more mutations in genes selected from CREBBP, KMT2D, BCL2, STAT6, NBPF1, EZH2, or a combination thereof, wherein the one or more mutations are listed in Table 8 or 10; or
d) the B-cell malignancy is RT and the method comprises analyzing a sample from the subject for one or more mutations in genes selected from ROS1, IGLL5, PASK, or a combination thereof, wherein the one or more mutations are listed in Table 12 or 14;

wherein the one or more mutations in the genes is indicative of nonresponsiveness to the combination.

In some embodiments, the B-cell malignancy is DLBCL and the method comprises analyzing a sample from the subject for one or more mutations in genes selected from TP53, EBF1, ADAMTS20, AKAP9, SOCS1, TNFRSF14, MYD88, NFKB1B, or a combination thereof, wherein the one or more mutations are listed in Table 4 or 6 and the one or more mutations in the genes is indicative of nonresponsiveness to the combination. One or more mutations as listed in Table 4 or 6 in 1, 2, 3, 4, 5, 6, 7, or all 8 of TP53, EBF1, ADAMTS20, AKAP9, SOCS1, TNFRSF14, MYD88, or NFKB1B and various combinations thereof can be indicative of nonresponsiveness to the combination.

In some embodiments, the B-cell malignancy is GCB-DLBCL and the method comprises analyzing a sample from the subject for one or more mutations in genes selected from KMT2D, BCL2, CSMD3, CREBBP, EBF1, SGK1, or a combination thereof, wherein the one or more mutations are listed in Table 16 and the one or more mutations in the genes is indicative of nonresponsiveness to the combination. One or more mutations as listed in Table 16 in 1, 2, 3, 4, 5, or all 6 of KMT2D, BCL2, CSMD3, CREBBP, EBF1, or SGK1 and various combinations thereof can be indicative of nonresponsiveness to the combination.

In some embodiments, the B-cell malignancy is FL and the method comprises analyzing a sample from the subject for one or more mutations in genes selected from CREBBP, KMT2D, BCL2, STAT6, NBPF1, EZH2, or a combination thereof, wherein the one or more mutations are listed in Table 8 or 10 and the one or more mutations in the genes is indicative of nonresponsiveness to the combination. One or more mutations as listed in Table 8 or 10 in 1, 2, 3, 4, 5, or all 6 of CREBBP, KMT2D, BCL2, STAT6, NBPF1, or EZH2 and various combinations thereof can be indicative of nonresponsiveness to the combination.

In some embodiments, the B-cell malignancy is RT and the method comprises analyzing a sample from the subject for one or more mutations in genes selected from ROS1, IGLL5, PASK, or a combination thereof, wherein the one or more mutations are listed in Table 12 or 14 and the one or more mutations in the genes is indicative of nonresponsiveness to the combination. In some aspects, the B-cell malignancy is RT and the method comprises analyzing a sample from the subject for one or more mutations in ROS1, wherein the one or more mutations are listed in Table 12 or 14. One or more mutations as listed in Table 12 or 14 in 1, 2, or all 3 of ROS1, IGLL5, or PASK and various combinations thereof can be indicative of nonresponsiveness to the combination.

Suitable samples from the subject include, for example, blood or tumor samples.

The disclosed methods can be used to predict the likelihood of responsiveness or nonresponsiveness to the combination in subjects who:

a) have DLBCL, FL, or RT (transformation from CLL/SLL only);

b) had ≥1 prior therapy (≥2 prior therapies for FL) but no more than 4 prior lines of treatment;

c) had an Eastern Cooperative Oncology Group (ECOG) performance status ≤2;

d) have measurable disease; and

e) had no prior ibrutinib or anti-PD-1 therapies.

In some embodiments, the methods of predicting a likelihood of responsiveness or nonresponsiveness to a combination of ibrutinib and an anti-PD-1 antibody further comprises administering a therapeutically effective amount of the combination of ibrutinib and an anti-PD-1 antibody to the subject to thereby treat the B-cell malignancy if the subject has one or more mutations in genes that are indicative of responsiveness to the combination and/or a lack one or more mutations in genes that are indicative of nonresponsiveness to the combination, the one or more mutations listed in Tables 4, 6, 8, 10, 12, 14, and 16. In some aspects, the anti-PD-1 antibody comprises nivolumab (brand name OPDIVO®).

Suitable amounts of ibrutinib, amounts of the anti-PD-1 antibody, and dosing schedules include those disclosed above for the methods of treatment.

### EXAMPLES

The following examples are provided to further describe some of the embodiments disclosed herein. The examples are intended to illustrate, not to limit, the disclosed embodiments.

Genetic Analyses of Subjects Having Relapsed Diffuse Large B-Cell Lymphoma (DLBCL), Follicular Lymphoma (FL), or Richter's Transformation (RT) Treated with Ibrutinib+Nivolumab

A phase 1/2a study (referred to as LYM1002) was performed to investigate the use of ibrutinib combined with the anti-PD-1 agent nivolumab in patients with relapsed or refractory (R/R) B-cell malignancies and to identify predictive and mechanistic genes correlated with response.

### Methods

### Patients and Study Design

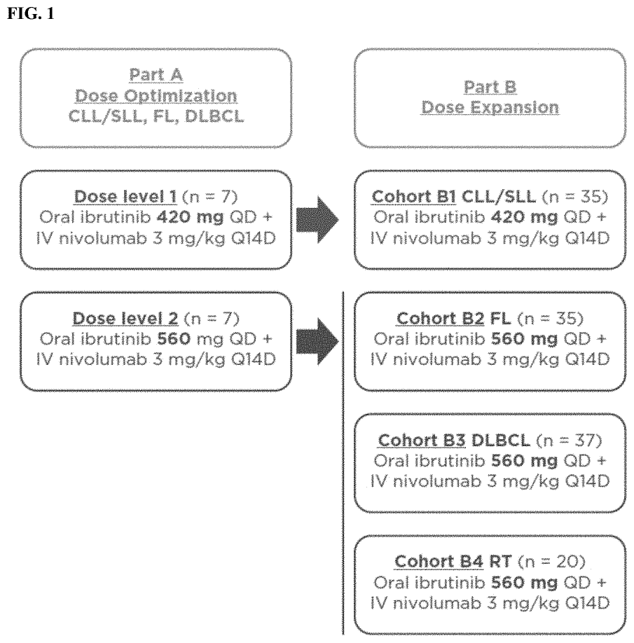

This nonrandomized, open-label trial enrolled patients with non-Hodgkin's lymphoma (NHL) who received intravenous (IV) nivolumab (3 mg/kg) on a 14-day cycle combined with oral ibrutinib (560 mg) once daily (FIG. 1). Key eligibility criteria were:
DLBCL, FL, or RT (transformation from CLL/SLL only);
≥1 prior systemic therapy (≥2 for FL) but no more than 4 prior lines of treatment;
Eastern Cooperative Oncology Group (ECOG) performance status ≤2;
Measurable disease; and
No prior ibrutinib or anti-PD-1 therapy.

Patients were excluded for major surgery within 4 weeks of the first dose of ibrutinib, diagnosis or treatment of malignancies other than the indication under study, or requiring treatment with warfarin or equivalent vitamin K antagonists or strong CYP3A inhibitors. Biomarker analyses were conducted in patients with DLBCL, FL, and RT.

### Assessments

DLBCL subtyping—gene expression profiling (GEP) was performed using AffyMetrix HG-U133+2 arrays (Thermo Fisher Scientific, Carlsbad, Calif.) and RNA from archived biopsy samples prior to treatment. DLBCL subtyping was conducted either by analysis of MAS5-normalized GEP data using the classification algorithm described in Wright G, Tan B, Rosenwald A, Hurt E H, Wiestner A, Staudt L M. A gene expression-based method to diagnose clinically distinct subgroups of diffuse large B cell lymphoma. Proc Natl Acad Sci USA 2003; 100(17): 9991-6 or the HTG system (HTG Molecular Diagnostics, Inc., Tucson, Ariz.).

Treatment response and survival outcomes—Preliminary activity and clinical response to treatment were evaluated by radiological assessments every five cycles (14-day cycles) for the first 15 months and every 12 cycles thereafter until disease progression, at the end of treatment, and every six months during the follow-up period. For calculation of overall response rate (ORR), responders were defined as patients who achieved complete response (CR) or partial response (PR) by investigator assessment. Progression-free survival (PFS) and overall survival (OS) were estimated using the Kaplan-Meier method and log-rank test.

### Clinical Outcome Analyses by Biomarker

PD-L1 expression—PD-L1 expression as a predictive biomarker for clinical outcomes was evaluated. PD-L1 levels were identified using GEP, and also as the percentage of tumor cells demonstrating plasma membrane PD-L1 staining of any intensity in a minimum of 100 evaluable tumor cells using the Dako PD-L1 IHC 28-8 pharmDx assay (Agilent Technologies, Glostrup, Denmark). GEP was performed using AffyMetrix HG-U133+2 arrays and RNA from archived biopsy samples prior to treatment.

Kaplan-Meier survival probability with response or survival endpoints was calculated for patients with elevated or nonelevated PD-L1 subgroups with DLBCL, FL, and RT, using the immunohistochemistry (IHC)HC threshold of ≥5% PD-L1 expression in tumor cells (elevated vs. nonelevated). The association of PD-L1 with clinical response was assessed using Fisher's exact test. DLBCL subtyping was conducted either by analysis of MAS5-normalized GEP data using the Sensation Method or by using the HTG EdgeSeq system. PD-L1 levels were measured by IHC staining using the Dako 28-8 antibody (PD-L1 elevation=expression in ≥5% of tumor cells).

Responders were defined as patients who achieved complete response (CR) or partial response (PR). Progression-free survival (PFS) and overall survival (OS) were evaluated using the Kaplan-Meier method and log-rank test.

Exome analyses—Exome data were generated from formalin-fixed paraffin embedded samples of 72 lymphoma samples, each from a different patient. An in-house exome analysis pipeline was run on DNAnexus using raw FASTQ sequence data files. Likely somatic variants were defined based on annotations made with SnpEff and GEMINI software. A number of variant filters were put in place to reduce the likelihood of incorporating sequencing artifacts and germline variants into the association analysis.

The incidence of mutations was assessed for specific genes of interest, including those from the Personalis ACE Extended Cancer panel, DLBCL-associated genes (i.e., ABC/GCB discriminating genes, genes used to discriminate between four newly defined subtypes, genes predicted as hypermutated in DLBCL), and a Janssen-specific 97-gene panel.

Any differences between treatment responders (CR+PR+PR−L) and nonresponders (no response or SD+SD+PD), and between patients with ongoing responses (PFS >24 months) vs. not, were investigated for mutational variants, gene expression patterns, and somatic mutation burden. Univariate gene analysis examined the significance of variant frequencies for responders vs. nonresponders and PFS >24 months vs. not using Fisher's exact test. Differential gene expression analyses for responders vs. nonresponders and PFS >24 months vs. not were performed using the “limma” R package. Overall differences in somatic mutation counts for responders vs. nonresponders and patients with PFS >24 months vs. not were assessed using the Wilcoxon signed-rank test.

### Patients and Clinical Responses

Of 144 subjects enrolled, 141 received treatment. For these patients, the median age was 65 years (range 20-89 years), 87 (61.7%) were male, 130 (92.2%) had an ECOG performance status of 0-1, with a median of 3 prior lines of therapy, and 68 (48.2%) had bulky disease (≥5 cm).

Overall, 45 patients with DLBCL (9 with transformed DLBCL and 36 de novo DLBCL), 40 with FL, and 20 with RT were enrolled. Of these, 28 patients with DLBCL (4 transformed), 25 with FL, and 17 with RT were evaluable for genes by GEP analysis.

The overall median follow-up at the time of database lock was 19.4 months (range 0.4-28.8 months).

In patients with GEP data, overall response rates were 29.6% for DLBCL, 43.5% for FL, and 81.3% for RT (Table 1).

TABLE 1
Response to DLBCL, FL, and RT patients with GEP data

| | DLBCL | | | | |
|---|---|---|---|---|---|
| | Total | ABC | GCB | FL | RT |
| Population, n | 28 | 5 | 19 | 25 | 17 |
| Overall response | 8 | 2 | 6 | 10 | 13 |
| rate (R/R + NR), % | (29.6) | (40) | (33.3) | (43.5) | (81.3) |
| Responders, n | 8 | 2 | 6 | 10 | 13 |
| (% of total) | (29.6) | (40) | (33.3) | (43.5) | (81.3) |
| CR, n | 4 | 2 | 2 | 3 | 2 |
| (% of total) | (14.8) | (40) | (11.1) | (13.1) | (12.5) |
| PR, n | 4 | 0 | 4 | 7 | 11 |
| (% of total) | (14.8) | | (22.2) | (30.4) | (68.8) |
| Nonresponders, n | 19 | 3 | 12 | 13 | 3 |
| (% of total) | (70.4) | (50) | (66.7) | (56.5) | (18.8) |
| No response or SD, | 4 | 0 | 2 | 6 | 0 |
| n (% of total) | (14.8) | | (11.1) | (26.1) | |
| PD, n | 15 | 3 | 10 | 7 | 3 |
| (% of total) | (55.6) | (50) | (55.6) | (30.4) | (18.8) |
| Missing, n | 1 | 0 | 1 | 2 | 1 |

ABC = activated B-cell;
GCB = germinal center B-cell

### Subtyping

Patient subtypes were evaluated by GEP microarray and an HTG EdgeSeq DLBCL Cell of Origin (COO) Assay (HTG) method. 28 DLBCL patients were evaluable for subtyping using the GEP microarray method: 5 patients had the activated B-cell (ABC) subtype, 19 had the germinal center B-cell (GCB) subtype, and 4 were unclassified (Table 1). 13 DLBCL patients were evaluable for subtyping using the HTG method: 6 patients had the ABC subtype, 6 had the GCB subtype, and 1 was unclassified. Concordance between GEP and HTG methods was high—only 1 patient with DLBCL who was classified as GCB by GEP was subtyped as ABC by HTG.

### Pd-L1 Analysis

### PD-L1 Expression and Clinical Outcomes in DLBCL Patients

PD-L1 elevation (≥5% tumor cells) occurred in 8 (30.8%) DLBCL patients (3 CR, 2 PR), 1 (4.0%) FL patient, and 3 (20.0%) RT patients (all PR) (Table 2). Of DLBCL patients for which both PD-L1 IHC and GEP were available, 4/17 GCB (1 CR, 2 PR, 1 SD), 1/3 ABC (PD), and 1/3 intermediate (PD) patients had PD-L1 elevation.

In DLBCL, elevated PD-L1 was observed more frequently in responders versus nonresponders, although this was not statistically significant overall (62.5% vs 18.8%, p=0.06); elevated PD-L1 was also significantly associated with CR (37.5% vs 0; p=0.03 [Fisher exact test]).

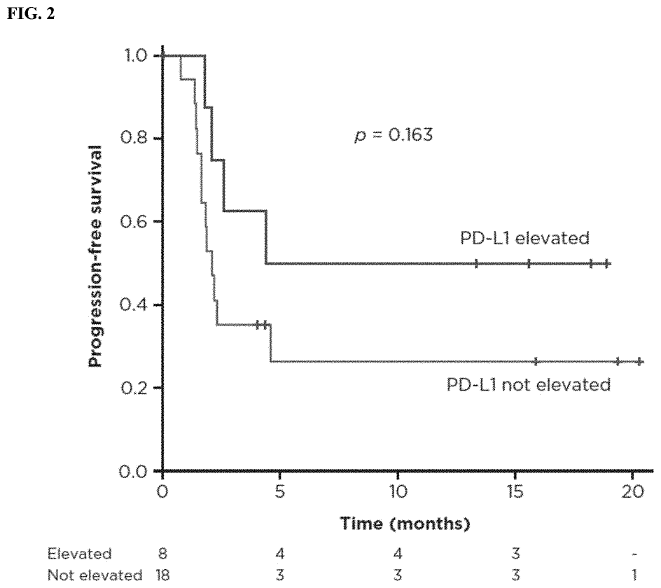

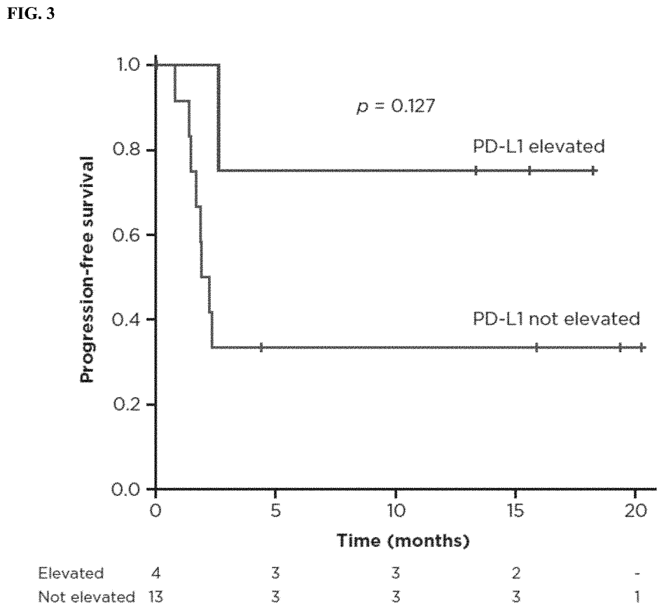

There was a trend toward improved PFS in DLBCL patients (n=26) (FIG. 2), as well as in GCB-DLBCL subtype (n=17) patients (FIG. 3) with elevated PD-L1 compared with those without elevation.

TABLE 2
PD-L1 expression by IHC and tumor type*

| | DLBCL | | | | |
|---|---|---|---|---|---|
| | Total | ABC | GCB | FL | RT |
| PD-L1 IHC expression | 8 | 1 | 4 | 1 | 3 |
| elevated (≥5%) | | | | | |
| PD-L1 IHC expression | 18 | 2 | 13 | 24 | 12 |
| not elevated (<5%) | | | | | |

*The patient numbers varied slightly between the different results based on the assay under consideration.

### PD-L1 Response and Survival in FL and RT Patients

A trend toward improved PFS could not be evaluated in FL patients, as only 1 FL patient was positive for PD-L1 by IHC. In RT, 13/16 evaluable patients responded, but only 3/15 patients with IHC data had elevated PD-L1 levels; all patients with elevated PD-L1 achieved PR. All 3 of these patients had durable PFS and OS and were alive at the time of clinical cutoff, but no significant correlations were possible due to the low numbers.

### Conclusions

In this study, DLBCL patients with elevated PD-L1 expression showed a trend toward better response and survival with ibrutinib and nivolumab treatment, although patient numbers were small and significance was reached only for CR.

The safety profile of the ibrutinib and nivolumab treatment was comparable with single-agent ibrutinib, and the overall response rate (ORR) was 32.5% for follicular lymphoma (FL), 35.6% for diffuse large B-cell lymphoma (DLBCL), and 65.0% for Richter's transformation (RT).

Of the 27 patients with DLBCL who had evaluable GEP data and responder/nonresponder status, the ORR was 29.6%, but most of these were the GCB subtype (ORR 33.3%), in which only an ORR of 5% was previously reported with single-agent ibrutinib. There were too few ABC subtype patients to permit robust analysis.

Clinical response in RT (who historically have had poor outcomes with single-agent ibrutinib or chemotherapy) exceeded expectation: ORR was 65.0% in patients who were screened and received treatment and 81.3% in patients with GEP data; although only 3 patients had elevated PD-L1 by IHC, all 3 had durable PR.

PD-1 typically helps concentrate Tfh cells in GCs by restricting CXCR3 expression on Tfh cells. The results herein suggest that there may be a distinct subset of GCB-DLBCL patients for whom the disease is primarily driven by Tfh cell activity; in these patients, anti-PD-1 therapy would likely decrease the proliferation and maturation of malignant B cells in the GC by inhibiting PD-L1/PD-1 interactions between Tfh and B cells.

### Exome and Sequence Analysis

A genetic analysis was performed using archived biopsy samples from subjects receiving the combination of ibrutinib and nivolumab. Exome data were generated from 72 formalin-fixed paraffin-embedded samples, and sequencing analysis was used to identify mutations in genes of interest and assess somatic mutation burden. The correlations of immune cell proportions and gene variants were evaluated by investigator-assessed responses in each histology and by ongoing responses in DLBCL patients (progression-free survival [PFS] >24 months, n=7 vs not, n=20). Overall response rate (ORR) was evaluated.

Responders Vs. Non-Responders

Gene variant and response data were available for 26 patients with DLBCL (10 responders (5 CR, 5 PR), 16 nonresponders), 16 patients with GCB DLBCL (6 responders (2 CR, 4 PR), 10 nonresponders), 26 patients with FL (12 responders (3 CR, 9 PR), 14 nonresponders), and 17 patients with RT (13 responders (2 CR, 11 PR), 4 nonresponders). The results are provided in Tables 3-16.

Tables 3 and 4 below provide mutation frequencies and specific gene mutations of the genes more frequently mutated in either responders or non-responders with DLBCL, with significance evaluated using the Fisher's exact test.

TABLE 3
Response data in DLBCL patients for genes
chosen based on Fisher's exact test results

| | | Non- | | |
|---|---|---|---|---|
| Gene | Responder | Responder | Odds Ratio (95 CI) | P-value* |
| KLHL14 | 3/10 | 0/16 | Inf (0.730, Inf) | 0.046 |
| | (30.0%) | (0.0%) | | |
| RNF213 | 4/10 | 1/16 | 9.053 (0.711, 522.371) | 0.055 |
| | (40.0%) | (6.2%) | | |
| EBF1 | 0/10 | 4/16 | 0.000 (0.000, 2.304) | 0.136 |
| | (0.0%) | (25.0%) | | |
| CAMTA1 | 2/10 | 0/16 | Inf (0.311, Inf) | 0.138 |
| | (20.0%) | (0.0%) | | |
| DIDO1 | 2/10 | 0/16 | Inf (0.311, Inf) | 0.138 |
| | (20.0%) | (0.0%) | | |
| GIGYF2 | 2/10 | 0/16 | Inf (0.311, Inf) | 0.138 |
| | (20.0%) | (0.0%) | | |
| NACA | 2/10 | 0/16 | Inf (0.311, Inf) | 0.138 |
| | (20.0%) | (0.0%) | | |
| SELP | 2/10 | 0/16 | Inf (0.311, Inf) | 0.138 |
| | (20.0%) | (0.0%) | | |
| ZMYM4 | 2/10 | 0/16 | Inf (0.311, Inf) | 0.138 |
| | (20.0%) | (0.0%) | | |

*Fisher's exact test results

TABLE 4
Gene variants in DLBCL patients for genes
chosen based on Fisher's exact test results

| | | | Codon | AA | Response |
|---|---|---|---|---|---|
| Gene | Transcript ID | Allele | change | change | group |
| CAMTAI | ENST00000303635 | C/T | gCg/gTg | A385V | Responder |
| CAMTAI | ENST00000303635 | G/A | Gac/Aac | D486N | Responder |
| DIDOI | ENST00000266070 | C/A | caG/caT | Q1539H | Responder |
| DIDOI | ENST00000266070 | G/A | Cga/Tga | R1835* | Responder |
| EBF1 | ENST00000313708 | G/A | Cgc/Tgc | R163C | Non-responder |
| EBF1 | ENST00000313708 | T/C | gAa/gGa | E17G | Non-responder |
| EBF1 | ENST00000313708 | A/G | Tgt/Cgt | C164R | Non-responder |
| EBF1 | ENST00000313708 | A/T | aTg/aAg | M232K | Non-responder |
| GIGYF2 | ENST00000421778 | A/G | Atg/Gtg | M1V | Responder |
| GIGYF2 | ENST00000452341 | A/G | Aga/Gga | R851G | Responder |
| KLHL14 | ENST00000583263 | G/A | Cca/Tca | P20S | Responder |
| KLHL14 | ENST00000359358 | C/A | gaG/gaT | E140D | Responder |
| KLHL14 | ENST00000359358 | T/C | Aac/Gac | N124D | Responder |
| KLHL14 | ENST00000359358 | C/A | aaG/aaT | K187N | Responder |
| KLHL14 | ENST00000359358 | C/T | cGc/cAc | R452H | Responder |
| NACA | ENST00000454682 | G/C | Cag/Gag | Q55E | Responder |
| NACA | ENST00000454682 | C/A | aGg/aTg | R502M | Responder |
| RNF213 | ENST00000508628 | G/C | Gaa/Caa | E4942Q | Responder |
| RNF213 | ENST00000508628 | C/T | gCc/gTc | A2744V | Responder |
| RNF213 | ENST00000508628 | C/A | Ctc/Atc | L4751I | Responder |
| RNF213 | ENST00000508628 | G/A | cGt/cAt | R4252H | Responder |
| SELP | ENST00000263686 | C/A | Gtg/Ttg | V758L | Responder |
| SELP | ENST00000263686 | G/A | tCg/tTg | S385L | Responder |
| ZMYM4 | ENST00000314607 | G/A | Gac/Aac | D1541N | Responder |
| ZMYM4 | ENST00000314607 | A/G | Act/Gct | T313A | Responder |

*Stop codon gained.

Tables 5 and 6 below provide mutation frequencies and specific gene mutations of the most frequently mutated genes in either responders or non-responders with DLBCL.

TABLE 5
Response data in DLBCL patients for genes chosen based on having a
high frequency of variants in either responders or non-responders

| Gene | Responder | Non-Responder |
|---|---|---|
| RNF213 | 4/10 | 1/16 |
| | (40.0%) | (6.2%) |
| CSMD3 | 3/10 | 7/16 |
| | (30.0%) | (43.8%) |
| BCL2 | 3/10 | 6/16 |
| | (30.0%) | (37.5%) |
| NBPF1 | 3/10 | 4/16 |
| | (30.0%) | (25.0%) |
| LRP1B | 3/10 | 1/16 |
| | (30.0%) | (6.2%) |
| TP53 | 0/10 | 3/16 |
| | (0.0%) | (18.8%) |
| TNFRSF14 | 0/10 | 3/16 |
| | (0.0%) | (18.8%) |
| ADAMTS20 | 0/10 | 3/16 |
| | (0.0%) | (18.8%) |
| AKAP9 | 0/10 | 3/16 |
| | (0.0%) | (18.8%) |
| SOCS1 | 0/10 | 3/16 |
| | (0.0%) | (18.8%) |
| MYD88 | 0/10 | 2/16 |
| | (0.0%) | (12.5%) |
| NFKBIB | 0/10 | 2/16 |
| | (0.0%) | (12.5%) |

TABLE 6
Gene variants in DLBCL patients for genes chosen based on having a
high frequency of variants in either responders or non-responders

| | | | Codon | AA | Response |
|---|---|---|---|---|---|
| Gene | Transcript ID | Allele | change | Change | group |
| BCL2 | ENST00000333681 | C/G | gGc/gCc | G47A | Responder |
| BCL2 | ENST00000333681 | T/A | tAc/tTc | Y28F | Responder |
| BCL2 | ENST00000333681 | G/T | gCc/gAc | A131D | Responder |
| BCL2 | ENST00000333681 | G/A | gCc/gTc | A77V | Responder |
| BCL2 | ENST00000333681 | G/A | aCc/aTc | T125I | Responder |
| BCL2 | ENST00000333681 | G/C | gCc/gGc | A113G | Responder |
| BCL2 | ENST00000333681 | G/A | Cac/Tac | H120Y | Responder |
| BCL2 | ENST00000333681 | T/C | Aca/Gca | T7A | Responder |
| BCL2 | ENST00000333681 | C/T | Gat/Aat | D34N | Responder |
| BCL2 | ENST00000589955 | C/T | Gca/Aca | A198T | Responder |
| CSMD3 | ENST00000297405 | C/A | Gtt/Ttt | V382F | Responder |
| CSMD3 | ENST00000297405 | C/G | Gtt/Ctt | V3667L | Responder |
| CSMD3 | ENST00000297405 | G/T | gCt/gAt | A1975D | Responder |
| CSMD3 | ENST00000297405 | G/C | cCa/cGa | P1475R | Responder |
| LRP1B | ENST00000389484 | A/T | Ttt/Att | F1575I | Responder |
| LRP1B | ENST00000389484 | A/T | taT/taA | Y4562* | Responder |
| LRP1B | ENST00000389484 | T/C | gAa/gGa | E4125G | Responder |
| MYD88 | ENST00000417037 | G/A | aGc/aAc | S251N | Non-responder |
| MYD88 | ENST00000495303 | T/C | Tga/Cga | 160R** | Non-responder |
| NBPF1 | ENST00000430580 | T/C | aAa/aGa | K41R | Responder |
| NBPF1 | ENST00000430580 | T/A | aAg/aTg | K623M | Responder |
| NBPF1 | ENST00000430580 | G/T | Ccc/Acc | P926T | Responder |
| NFKBIB | ENST00000313582 | C/T | Cgg/Tgg | R339W | Non-responder |
| NFKBIB | ENST00000313582 | C/T | cCg/cTg | P236L | Non-responder |
| RNF213 | ENST00000508628 | G/C | Gaa/Caa | E4942Q | Responder |
| RNF213 | ENST00000508628 | C/T | gCc/gTc | A2744V | Responder |
| RNF213 | ENST00000508628 | C/A | Ctc/Atc | L4751I | Responder |
| RNF213 | ENST00000508628 | G/A | cGt/cAt | R4252H | Responder |
| TNFRSF14 | ENST00000355716 | T/A | Tgc/Agc | C53S | Non-responder |
| TNFRSF14 | ENST00000355716 | G/A | tGc/tAc | C57Y | Non-responder |
| TNFRSF14 | ENST00000355716 | G/T | Gga/Tga | G5* | Non-responder |
| TP53 | ENST00000269305 | C/A | tGc/tTc | C135F | Non-responder |
| TP53 | ENST00000269305 | C/T | cGt/cAt | R273H | Non-responder |
| TP53 | ENST00000269305 | A/T | Ttt/Att | F1341 | Non-responder |
| ADAMTS20 | ENST00000389420 | C/T | cGc/cAc | R132H | Non-responder |
| ADAMTS20 | ENST00000389420 | C/A | gGa/gTa | G1836V | Non-responder |
| ADAMTS20 | ENST00000389420 | G/C | aaC/aaG | N1733K | Non-responder |
| ADAMTS20 | ENST00000389420 | T/G | aaA/aaC | K1684N | Non-responder |
| AKAP9 | ENST00000359028 | A/C | Agt/Cgt | S2451R | Non-responder |
| AKAP9 | ENST00000359028 | A/C | gAg/gCg | E775A | Non-responder |
| AKAP9 | ENST00000359028 | G/A | aGc/aAc | S2789N | Non-responder |
| SOCS1 | ENST00000332029 | G/A | Ccc/Tcc | P97S | Non-responder |
| SOCS1 | ENST00000332029 | G/C | agC/agG | S143R | Non-responder |
| SOCS1 | ENST00000332029 | T/A | aAc/aTc | N5I | Non-responder |
| SOCS1 | ENST00000332029 | C/G | aGc/aCc | S116T | Non-responder |
| SOCS1 | ENST00000332029 | C/A | Gca/Tca | A3S | Non-responder |

*Stop codon gained; **start codon lost.

Responder vs. nonresponder—In DLBCL, the most frequent gene variants observed in responders included KLHL14 (n=3), RNF213 (n=4), CSMD3 (n=3), BCL2 (n=3), NBPF1 (n=3), and LRP1B (n=3). Conversely, the most frequent gene variants observed in nonresponders included TP53 (n=3), EBF1 (n=4), ADAMTS20 (n=3), AKAP9 (n=3), and SOCS1 (n=3), and genes in BCR pathways such as TNFRSF14 (n=3), MYD88 (n=2), and NFKB1B (n=2). The greatest differences in gene variant frequency between responders and nonresponders were seen for KLHL14 mutations (3/10 (30.0%) vs. 0/16; odds ratio (OR) (95% confidence interval (CI)) inf [0.730-inf]; P=0.046) and RNF213 mutations (4/10 (40.0%) vs. 1/16 (6.2%); OR (95% CI) 9.053 (0.711-522.371); P=0.055). Thus, in DLBCL patients, those with RNF213 and KLHL14 mutations were more likely to respond to ibrutinib+nivolumab.

Tables 7 and 8 below provide mutation frequencies and specific gene mutations of the genes more frequently mutated in either responders or non-responders with FL, with significance evaluated using the Fisher's exact test.

TABLE 7
Response data in FL patients for genes chosen
based on Fisher's exact test results

| Gene | Responder | Non-Responder | Odds Ratio (95 CI) | | P-value* |
|---|---|---|---|---|---|
| BCL2 | 9/12 | 4/14 | 6.847 | (1.019, 62.695) | 0.047 |
| | (75.0%) | (28.6%) | | | |
| CIITA | 3/12 | 0/14 | Inf | (0.515, Inf) | 0.085 |
| | (25.0%) | (0.0%) | | | |
| FES | 3/12 | 0/14 | Inf | (0.515, Inf) | 0.085 |
| | (25.0%) | (0.0%) | | | |
| NCOA2 | 3/12 | 0/14 | Inf | (0.515, Inf) | 0.085 |
| | (25.0%) | (0.0%) | | | |
| TPR | 3/12 | 0/14 | Inf | (0.515, Inf) | 0.085 |
| | (25.0%) | (0.0%) | | | |
| NBPF1 | 0/12 | 4/14 | 0.000 | (0.000, 1.615) | 0.1 |
| | (0.0%) | (28.6%) | | | |

*Fisher's exact test results

TABLE 8
Gene variants in FL patients for genes chosen based on Fisher's exact test results

| | | | Codon | AA | Response |
|---|---|---|---|---|---|
| Gene | Transcript ID | Allele | change | change | group |
| BCL2 | ENST000005 89955 | C/T | aGt/aAt | S203N | Responder |
| BCL2 | ENST00000333681 | T/C | Agc/Ggc | S87G | Responder |
| BCL2 | ENST00000333681 | A/G | gTg/gCg | V159A | Responder |
| BCL2 | ENST00000333681 | G/A | Cca/Tca | P59S | Responder |
| BCL2 | ENST00000333681 | C/T | Gcg/Acg | A2T | Responder |
| BCL2 | ENST00000333681 | C/T | Gcg/Acg | A85T | Responder |
| BCL2 | ENST00000333681 | C/T | cGc/cAc | R129H | Responder |
| BCL2 | ENST00000333681 | C/G | Gct/Cct | A4P | Responder |
| BCL2 | ENST00000333681 | T/A | cAg/cTg | Q190L | Responder |
| BCL2 | ENST00000589955 | C/G | Ggt/Cgt | G197R | Responder |
| BCL2 | ENST00000333681 | G/A | cCa/cTa | P59L | Responder |
| BCL2 | ENST00000333681 | G/A | gCc/gTc | A60V | Responder |
| BCL2 | ENST00000333681 | G/A | Ccg/Tcg | P46S | Responder |
| CIITA | ENST00000324288 | C/T | Cca/Tca | P292S | Responder |
| CIITA | ENST00000324288 | C/T | Ccc/Tcc | P16S | Responder |
| CIITA | ENST00000324288 | C/A | Cct/Act | P952T | Responder |
| FES | ENST00000328850 | G/A | cGg/cAg | R246Q | Responder |
| FES | ENST00000328850 | A/T | Atc/Ttc | 1431F | Responder |
| FES | ENST00000328850 | C/T | Cgg/Tgg | R191W | Responder |
| NBPF1 | ENST00000430580 | T/A | aAg/aTg | K623M | Non-responder |
| NCOA2 | ENST00000452400 | G/C | cCt/cGt | P673R | Responder |
| NCOA2 | ENST00000452400 | T/C | atA/atG | I427M | Responder |
| NCOA2 | ENST00000452400 | T/C | Agt/Ggt | S420G | Responder |
| NCOA2 | ENST00000452400 | T/C | aAc/aGc | N401S | Responder |
| NCOA2 | ENST00000452400 | T/C | Acg/Gcg | T390A | Responder |
| NCOA2 | ENST00000452400 | C/A | aGt/aTt | S698I | Responder |
| TPR | ENST00000367478 | G/C | Cag/Gag | Q2287E | Responder |
| TPR | ENST00000367478 | T/C | aAa/aGa | K315R | Responder |
| TPR | ENST00000367478 | G/C | caC/caG | H2029Q | Responder |
| TPR | ENST00000367478 | T/G | Aat/Cat | N2028H | Responder |
| TPR | ENST00000367478 | C/T | Ggt/Agt | G2027S | Responder |
| TPR | ENST00000367478 | C/G | gGt/gCt | G2025A | Responder |
| TPR | ENST00000367478 | C/G | aGt/aCt | S1042T | Responder |

Tables 9 and 10 below provide mutation frequencies and specific gene mutations of the most frequently mutated genes in either responders or non-responders with FL.

TABLE 9
Response data in FL patients for genes chosen based on having a
high frequency of variants in either responders or non-responders

| Gene | Responder | Non-Responder |
|---|---|---|
| BCL2 | 9/12 | 4/14 |
| | (75.0%) | (28.6%) |
| CREBBP | 7/12 | 9/14 |
| | (58.3%) | (64.3%) |
| EZH2 | 1/12 | 4/14 |
| | (8.3%) | (28.6%) |
| KMT2D | 6/12 | 5/14 |
| | (50.0%) | (35.7%) |
| MUC17 | 4/12 | 3/14 |
| | (33.3%) | (21.4%) |
| NBPF1 | 0/12 | 4/14 |
| | (0.0%) | (28.6%) |
| STAT6 | 1/12 | 4/14 |
| | (8.3%) | (28.6%) |

TABLE 10
Gene variants in FL patients for genes chosen based on having a
high frequency of variants in either responders or non-responders

| | | | Codon | AA | Response |
|---|---|---|---|---|---|
| Gene | Transcript ID | Allele | change | Change | group |
| BCL2 | ENST00000333681 | C/G | gGc/gCc | G36A | Non-responder |
| BCL2 | ENST00000333681 | G/C | Ctg/Gtg | L119V | Non-responder |
| BCL2 | ENST00000589955 | C/T | aGt/aAt | S203N | Responder |
| BCL2 | ENST00000333681 | T/C | Agc/Ggc | S87G | Responder |
| BCL2 | ENST00000333681 | A/G | gTg/gCg | V159A | Responder |
| BCL2 | ENST00000333681 | G/A | Cca/Tca | P59S | Responder |
| BCL2 | ENST00000333681 | C/T | Gcg/Acg | A2T | Responder |
| BCL2 | ENST00000333681 | C/T | Gcg/Acg | A85T | Responder |
| BCL2 | ENST00000589955 | C/T | Ggt/Agt | G197S | Non-responder |
| BCL2 | ENST00000333681 | G/A | Cac/Tac | H120Y | Non-responder |
| BCL2 | ENST00000333681 | C/G | aGa/aCa | R6T | Non-responder |
| BCL2 | ENST00000589955 | C/G | aGt/aCt | S203T | Non-responder |
| BCL2 | ENST00000333681 | T/C | Acc/Gcc | T187A | Non-responder |
| BCL2 | ENST00000333681 | G/C | Cca/Gca | P59A | Non-responder |
| BCL2 | ENST00000333681 | C/T | cGc/cAc | R129H | Responder |
| BCL2 | ENST00000333681 | T/C | aAg/aGg | K239R | Non-responder |
| BCL2 | ENST00000333681 | C/T | Gat/Aat | D191N | Non-responder |
| BCL2 | ENST00000333681 | G/C | Cag/Gag | Q52E | Non-responder |
| BCL2 | ENST00000333681 | G/A | aCa/aTa | T7I | Non-responder |
| BCL2 | ENST00000333681 | G/A | Ccc/Tcc | P53S | Non-responder |
| BCL2 | ENST00000333681 | C/G | Gct/Cct | A4P | Responder |
| BCL2 | ENST00000333681 | T/A | cAg/cTg | Q190L | Responder |
| BCL2 | ENST00000589955 | C/G | Ggt/Cgt | G197R | Responder |
| BCL2 | ENST00000333681 | G/A | cCa/cTa | P59L | Responder |
| BCL2 | ENST00000333681 | G/A | gCc/gTc | A60V | Responder |
| BCL2 | ENST00000333681 | G/A | Ccg/Tcg | P46S | Responder |
| CREBBP | ENST00000262367 | G/C | Cgc/Ggc | R1664G | Non-responder |
| CREBBP | ENST00000262367 | G/A | Cga/Tga | R1498* | Non-responder |
| CREBBP | ENST00000262367 | G/A | Cag/Tag | Q540* | Responder |
| CREBBP | ENST00000262367 | T/A | Aaa/Taa | K1060* | Responder |
| CREBBP | ENST00000262367 | T/A | gAt/gTt | D1543V | Non-responder |
| CREBBP | ENST00000262367 | G/A | cCt/cTt | P1053L | Non-responder |
| CREBBP | ENST00000262367 | A/G | cTg/cCg | L1499P | Responder |
| CREBBP | ENST00000262367 | C/G | caG/caC | Q1259H | Non-responder |
| CREBBP | ENST00000262367 | C/A | cGc/cTc | R1446L | Non-responder |
| CREBBP | ENST00000262367 | G/A | Cga/Tga | R1341* | Non-responder |
| CREBBP | ENST00000262367 | A/C | Tac/Gac | Y1450D | Non-responder |
| CREBBP | ENST00000262367 | A/T | tgT/tgA | C398* | Non-responder |
| CREBBP | ENST00000262367 | A/T | Tac/Aac | Y1503N | Responder |
| CREBBP | ENST00000262367 | G/T | tgC/tgA | C1408* | Non-responder |
| CREBBP | ENST00000262367 | T/A | gAt/gTt | D1521V | Non-responder |
| CREBBP | ENST00000262367 | C/G | cGg/cCg | R2151P | Responder |
| CREBBP | ENST00000262367 | G/A | Caa/Taa | Q249* | Responder |
| CREBBP | ENST00000262367 | C/T | Ggc/Agc | G52S | Responder |
| CREBBP | ENST00000262367 | T/C | Acc/Gcc | T514A | Responder |
| CREBBP | ENST00000262367 | T/G | cAa/cCa | Q513P | Responder |
| CREBBP | ENST00000262367 | T/C | Aca/Gca | T462A | Responder |
| CREBBP | ENST00000262367 | A/C | Tac/Gac | Y1503D | Responder |
| CREBBP | ENST00000262367 | G/A | cCt/cTt | P1053L | Responder |
| EZH2 | ENST00000320356 | A/C | Ttt/Gtt | F670V | Non-responder |
| EZH2 | ENST00000320356 | A/T | Tac/Aac | Y646N | Non-responder |
| EZH2 | ENST00000320356 | G/A | gCa/gTa | A692V | Non-responder |
| EZH2 | ENST00000320356 | T/A | tAc/tTc | Y646F | Non-responder |
| KMT2D | ENST00000301067 | A/T | tTa/tAa | L957* | Non-responder |
| KMT2D | ENST00000301067 | G/A | Cag/Tag | Q3720* | Responder |
| KMT2D | ENST00000301067 | G/A | Cag/Tag | Q2004* | Responder |
| KMT2D | ENST00000301067 | G/A | Cag/Tag | Q1703* | Responder |
| KMT2D | ENST00000301067 | G/A | Cga/Tga | R2771* | Non-responder |
| KMT2D | ENST00000301067 | A/T | tTa/tAa | L3897* | Non-responder |
| KMT2D | ENST00000301067 | G/T | taC/taA | Y1771* | Non-responder |
| KMT2D | ENST00000301067 | G/C | tCa/tGa | S2312* | Non-responder |
| KMT2D | ENST00000301067 | G/A | Cag/Tag | Q4590* | Non-responder |
| KMT2D | ENST00000301067 | G/A | Cag/Tag | Q764* | Responder |
| KMT2D | ENST00000301067 | G/A | Cag/Tag | Q928* | Responder |
| KMT2D | ENST00000301067 | C/A | gaG/gaT | E1649D | Responder |
| KMT2D | ENST00000301067 | A/C | Ttg/Gtg | L1599V | Responder |
| KMT2D | ENST00000301067 | G/A | Caa/Taa | Q2796* | Non-responder |
| MUC17 | ENST00000306151 | A/C | Acc/Ccc | T1447P | Responder |
| MUC17 | ENST00000306151 | C/G | aCt/aGt | T2258S | Responder |
| MUC17 | ENST00000306151 | G/C | aGt/aCt | S546T | Responder |
| MUC17 | ENST00000306151 | C/G | cCc/cGc | P4014R | Responder |
| NBPF1 | ENST00000430580 | T/A | aAg/aTg | K623M | Non-responder |
| STAT6 | ENST00000300134 | C/A | Gac/Tac | D419Y | Non-responder |
| STAT6 | ENST00000300134 | T/C | gAc/gGc | D419G | Non-responder |
| STAT6 | ENST00000300134 | C/T | Gac/Aac | D419N | Non-responder |
| STAT6 | ENST00000300134 | C/G | Gat/Cat | D519H | Non-responder |

*Stop codon gained.

Responder vs. nonresponder—In patients with FL, the most frequent gene variants observed in responders were BCL2 (n=9), CREBBP (n=7), KMT2D (n=6), MUC17 (n=4), CIITA (n=3), FES (n=3), NCOA2 (n=3), and TPR (n=3). The most frequent gene variants observed in nonresponders were CREBBP (n=9), KMT2D (n=5), BCL2 (n=4), STAT6 (n=4), NBPF1 (n=4), and EZH2 (n=4). The difference in gene variant frequency between responders and nonresponders was significant for BCL2 (9/12 (75%) vs. 4/14 (28.6%); OR (95% CI) 6.847 (1.019-62.695); P=0.047).

Tables 11 and 12 below provide mutation frequencies and specific gene mutations of the genes more frequently mutated in either responders or non-responders with RT, with significance evaluated using the Fisher's exact test.

TABLE 11
Response data in RT patients for genes chosen
based on Fisher's exact test results

| Gene | Responder | Non-Responder | Odds Ratio (95 CI) | P-value* |
|---|---|---|---|---|
| ROS1 | 0/13 | 2/4 | 0.000 (0.000, 1.431) | 0.044 |
| | (0.0%) | (50.0%) | | |
| IGLL5 | 1/13 | 2/4 | 0.104 (0.001, 2.790) | 0.121 |
| | (7.7%) | (50.0%) | | |
| PASK | 1/13 | 2/4 | 0.104 (0.001, 2.790) | 0.121 |
| | (7.7%) | (50.0%) | | |

*Fisher's exact test results

TABLE 12
Gene variants in RT patients for genes chosen based on Fisher's exact test results

| | | | Codon | AA | Response |
|---|---|---|---|---|---|
| Gene | Transcript ID | Allele | change | Change | group |
| IGLL5 | ENST00000532223 | C/T | Ccc/Tcc | P75S | Non-responder |
| IGLL5 | ENST00000532223 | G/A | Gtt/Att | V56I | Non-responder |
| IGLL5 | ENST00000532223 | T/A | gTg/gAg | V8E | Non-responder |
| IGLL5 | ENST00000532223 | C/A | Cct/Act | P93T | Non-responder |
| IGLL5 | ENST00000532223 | G/A | Gag/Aag | E15K | Non-responder |
| IGLL5 | ENST00000532223 | C/A | Ctg/Atg | L39M | Non-responder |
| IGLL5 | ENST00000532223 | C/A | gCc/gAc | A32D | Non-responder |
| PASK | ENST00000358649 | C/T | tgG/tgA | W621* | Non-responder |
| PASK | ENST00000358649 | G/C | Cca/Gca | P779A | Non-responder |
| ROS1 | ENST00000368508 | G/C | Ctt/Gtt | L138V | Non-responder |
| ROS1 | ENST00000368508 | G/T | cCa/cAa | P1614Q | Non-responder |

*Stop codon gained.

Tables 13 and 14 below provide mutation frequencies and specific gene mutations of the most frequently mutated genes in either responders or non-responders with RT.

TABLE 13
Response data in RT patients for genes chosen based on having a
high frequency of variants in either responders or non-responders

| Gene | Responder | Non-Responder |
|---|---|---|
| KLHL6 | 3/13 | 0/4 |
| | (23.1%) | (0.0%) |
| SETX | 3/13 | 0/4 |
| | (23.1%) | (0.0%) |
| SF3B1 | 3/13 | 0/4 |
| | (23.1%) | (0.0%) |
| IRF2BP2 | 3/13 | 1/4 |
| | (23.1%) | (25.0%) |
| NBPF1 | 3/13 | 1/4 |
| | (23.1%) | (25.0%) |

TABLE 14
Gene variants in RT patients for genes chosen based on having a
high frequency of variants in either responders or non-responders

| | | | Codon | AA | Response |
|---|---|---|---|---|---|
| Gene | Transcript ID | Allele | change | change | group |
| IRF2BP2 | ENST00000366609 | G/A | Ccg/Tcg | P150S | Responder |
| IRF2BP2 | ENST00000366609 | G/A | gCc/gTc | A214V | Responder |
| IRF2BP2 | ENST00000366609 | G/A | Ctc/Ttc | L86F | Responder |
| KLHL6 | ENST00000341319 | A/G | aTg/aCg | M67T | Responder |
| KLHL6 | ENST00000341319 | A/G | cTg/cCg | L65P | Responder |
| KLHL6 | ENST00000341319 | A/G | aTg/aCg | M157T | Responder |
| KLHL6 | ENST00000341319 | G/C | Ctt/Gtt | L90V | Responder |
| NBPF1 | ENST00000430580 | T/A | aAg/aTg | K623M | Responder |
| NBPF1 | ENST00000430580 | C/T | cGt/cAt | R938H | Responder |
| SETX | ENST00000372169 | G/C | atC/atG | I787M | Responder |
| SETX | ENST00000372169 | G/A | aCc/aTc | T246I | Responder |
| SETX | ENST00000372169 | G/C | gCa/gGa | A1491G | Responder |
| SF3B1 | ENST00000335508 | T/C | tAt/tGt | Y765C | Responder |
| SF3B1 | ENST00000335508 | G/A | aCt/aTt | T663I | Responder |
| SF3B1 | ENST00000335508 | C/A | Gtt/Ttt | V701F | Responder |

Responder vs. nonresponder—In RT, the most frequent gene variants observed in responders included were IRF2BP2, NBPF1, KLHL6, SETX, and SF3B1 (all n=3), whereas the most frequent gene variants observed in nonresponders were ROS1, IGLL5, and PASK (all n=2). The difference in gene variant frequency between responders and nonresponders was significant for ROS1 (0/13 vs. 2/4 (50%); OR (95% CI) 0.000 (0.000-1.431); P=0.044).

Tables 15 and 16 below provide mutation frequencies and specific gene mutations of the most frequently mutated genes in either responders or non-responders with GCB-DLBCL.

TABLE 15
Response data in GCB-DLBCL patients for genes
chosen based on having a high frequency of variants
in either responders or non-responders

| | | |
|---|---|---|
| Gene | Responder | Non-responder |

| | | | |
|---|---|---|---|
| CSMD3 | 0/6 | (0.0%) | 5/10 (50.0%) |
| BCL2 | 1/6 | (16.7%) | 6/10 (60.0%) |
| KMT2D | 1/6 | (16.7%) | 6/10 (60.0%) |
| CREBBP | 0/6 | (0.0%) | 4/10 (40.0%) |
| EBF1 | 0/6 | (0.0%) | 4/10 (40.0%) |
| SGK1 | 0/6 | (0.0%) | 4/10 (40.0%) |
| RNF213 | 2/6 | (33.3%) | 1/10 (10.0%) |
| NBPF1 | 2/6 | (33.3%) | 1/10 (10.0%) |

TABLE 16
Gene variants in GCB-DLBCL patients for genes chosen based on having
a high frequency of variants in either responders or non-responders

| | | | Codon | AA | Response |
|---|---|---|---|---|---|
| Gene | Transcript ID | Allele | change | change | group |
| BCL2 | ENST00000333681 | A/G | gTc/gCc | V156A | Nonresponder |
| BCL2 | ENST00000333681 | G/C | aCa/aGa | T7R | Nonresponder |
| BCL2 | ENST00000333681 | C/T | Gat/Aat | D31N | Nonresponder |
| BCL2 | ENST00000333681 | T/C | Atc/Gtc | I48V | Nonresponder |
| BCL2 | ENST00000333681 | C/T | Gag/Aag | E165K | Nonresponder |
| BCL2 | ENST00000333681 | T/C | aAc/aGc | N143S | Nonresponder |
| BCL2 | ENST00000589955 | C/T | Gca/Aca | A198T | Nonresponder |
| BCL2 | ENST00000589955 | G/A | gCa/gTa | A198V | Nonresponder |
| BCL2 | ENST00000333681 | G/C | aaC/aaG | N163K | Nonresponder |
| BCL2 | ENST00000333681 | T/G | gAg/gCg | E179A | Nonresponder |
| BCL2 | ENST00000333681 | G/A | Ccc/Tcc | P53S | Nonresponder |
| BCL2 | ENST00000589955 | C/G | Ggt/Cgt | G197R | Nonresponder |
| CREBBP | ENST00000262367 | A/C | tTt/tGt | F1185C | Nonresponder |
| CREBBP | ENST00000262367 | A/C | cTt/cGt | L1181R | Nonresponder |
| CREBBP | ENST00000262367 | G/C | cCt/cGt | P227R | Nonresponder |
| CSMD3 | ENST00000297405 | A/T | tTg/tAg | L3207* | Nonresponder |
| CSMD3 | ENST00000297405 | A/G | aTg/aCg | M2445T | Nonresponder |
| CSMD3 | ENST00000297405 | C/A | Ggg/Tgg | G2318W | Nonresponder |
| CSMD3 | ENST00000297405 | G/T | aCa/aAa | T604K | Nonresponder |
| EBF1 | ENST00000313708 | G/A | Cgc/Tgc | R163C | Nonresponder |
| EBF1 | ENST00000313708 | T/C | gAa/gGa | E17G | Nonresponder |
| EBF1 | ENST00000313708 | A/G | Tgt/Cgt | C164R | Nonresponder |
| KMT2D | ENST00000301067 | G/A | Cga/Tga | R2099* | Nonresponder |
| KMT2D | ENST00000301067 | T/C | gAt/gGt | D5462G | Nonresponder |
| KMT2D | ENST00000301067 | G/A | Cag/Tag | Q3265* | Nonresponder |
| KMT2D | ENST00000301067 | C/A | cGg/cTg | R755L | Nonresponder |
| KMT2D | ENST00000301067 | G/A | gCc/gTc | A5212V | Nonresponder |
| KMT2D | ENST00000301067 | G/A | Caa/Taa | Q4322* | Nonresponder |
| NBPF1 | ENST00000430580 | T/C | aAa/aGa | K41R | Responder |
| NBPF1 | ENST00000430580 | T/A | aAg/aTg | K623M | Responder |
| NBPF1 | ENST00000430580 | G/T | Ccc/Acc | P926T | Responder |
| RNF213 | ENST00000508628 | G/C | Gaa/Caa | E4942Q | Responder |
| RNF213 | ENST00000508628 | C/T | gCc/gTc | A2744V | Responder |
| SGK1 | ENST00000237305 | T/G | Atc/Ctc | I25L | Nonresponder |
| SGK1 | ENST00000367858 | C/A | aGg/aTg | R127M | Nonresponder |
| SGK1 | ENST00000367857 | G/A | Ccg/Tcg | P3S | Nonresponder |

Responder vs. nonresponder—In GCB-DLBCL, the most frequent gene mutations observed in responders (n=6) included RNF213 (n=2) and NBPF1 (n=2). In nonresponders (n=10), they were KMT2D (n=6), BCL2 (n=6), CSMD3 (n=5), CREBBP (n=4), EBF1 (n=4), and SGK1 (n=4). There were no significant differences in gene variant frequencies between responders and nonresponders with the GCB subtype (data not shown).

Somatic mutation burden—No significant differences were observed in overall somatic mutation counts between responders and nonresponders with DLBCL, FL, or RT, though in GCB DLBCL the count was significantly lower in responders than nonresponders (P=0.003) (data not shown). The number of somatic mutation variants was significantly lower in patients with DLBCL and PFS >24 months vs. not (P=0.0288) (data not shown).

Progression Free Survival (PFS) Ongoing for Greater than (>) 24 Months

PFS ongoing for >24 months vs. not in DLBCL patients was analyzed. The results are provided in Tables 17 and 18 below.

TABLE 17
PFS24 mutation frequency data in DLBCL patients for genes
chosen based on having a high frequency of variants in
the either the set of patients having PFS ongoing for
>24 months or the set of patients with shorter PFS

| Gene | Ongoing24 | Not |
|---|---|---|
| BCL2 | 3/7 | 6/20 |
| | (42.9%) | (30.0%) |
| CSMD3 | 2/7 | 8/20 |
| | (28.6%) | (40.0%) |
| NBPF1 | 3/7 | 4/20 |
| | (42.9%) | (20.0%) |
| KMT2D | 1/7 | 8/20 |
| | (14.3%) | (40.0%) |
| RNF213 | 3/7 | 2/20 |
| | (42.9%) | (10.0%) |
| CREBBP | 0/7 | 6/20 |
| | (0.0%) | (30.0%) |

TABLE 18
Gene variants in DLBCL patients for genes chosen based on having a
high frequency of variants in the either the set of patients having
PFS ongoing for >24 months or the set of patients with shorter PFS

| | | | Codon | AA | Ongoing24 |
|---|---|---|---|---|---|
| Gene | Transcript ID | Allele | change | change | group |
| BCL2 | ENST00000333681 | G/T | aaC/aaA | N163K | Not |
| BCL2 | ENST00000333681 | A/G | gTc/gCc | V156A | Not |
| BCL2 | ENST00000333681 | G/C | aCa/aGa | T7R | Not |
| BCL2 | ENST00000589955 | C/T | Gca/Aca | A198T | Ongoing24 |
| BCL2 | ENST00000333681 | G/A | aCc/aTc | T125I | Ongoing24 |
| BCL2 | ENST00000333681 | G/A | Cac/Tac | H120Y | Ongoing24 |
| BCL2 | ENST00000333681 | G/C | gCc/gGc | A113G | Ongoing24 |
| BCL2 | ENST00000333681 | C/T | Gat/Aat | D34N | Ongoing24 |
| BCL2 | ENST00000333681 | T/C | Aca/Gca | T7A | Ongoing24 |
| BCL2 | ENST00000333681 | G/T | gCc/gAc | A131D | Ongoing24 |
| BCL2 | ENST00000333681 | G/A | gCc/gTc | A77V | Ongoing24 |
| BCL2 | ENST00000333681 | C/G | gGc/gCc | G47A | Ongoing24 |
| BCL2 | ENST00000333681 | T/A | tAc/tTc | Y28F | Ongoing24 |
| BCL2 | ENST00000333681 | G/A | Ccc/Tcc | P53S | Not |
| BCL2 | ENST00000333681 | T/C | Atc/Gtc | I48V | Not |
| BCL2 | ENST00000333681 | C/T | Gat/Aat | D31N | Not |
| BCL2 | ENST00000333681 | C/T | Gag/Aag | E165K | Ongoing24 |
| BCL2 | ENST00000333681 | C/T | Gct/Act | A76T | Not |
| BCL2 | ENST00000589955 | G/A | gCa/gTa | A198V | Not |
| BCL2 | ENST00000589955 | C/T | Gca/Aca | A198T | Not |
| BCL2 | ENST00000589955 | C/G | Ggt/Cgt | G197R | Not |
| BCL2 | ENST00000333681 | T/G | gAg/gCg | E179A | Not |
| BCL2 | ENST00000333681 | G/C | aaC/aaG | N163K | Not |
| BCL2 | ENST00000333681 | T/C | aAc/aGc | N143S | Not |
| CREBBP | ENST00000262367 | A/C | tTt/tGt | F1185C | Not |
| CREBBP | ENST00000262367 | A/C | cTt/cGt | L1181R | Not |
| CREBBP | ENST00000262367 | G/T | Caa/Aaa | Q1491K | Not |
| CREBBP | ENST00000262367 | G/C | cCt/cGt | P227R | Not |
| CREBBP | ENST00000262367 | T/A | Aaa/Taa | K1060* | Not |
| CSMD3 | ENST00000297405 | C/G | Gtt/Ctt | V3667L | Not |
| CSMD3 | ENST00000297405 | C/A | Gtt/Ttt | V382F | Not |
| CSMD3 | ENST00000297405 | G/T | Cac/Aac | H350N | Not |
| CSMD3 | ENST00000297405 | A/T | tTg/tAg | L3207* | Not |
| CSMD3 | ENST00000297405 | A/G | aTg/aCg | M2445T | Not |
| CSMD3 | ENST00000297405 | C/T | gGc/gAc | G609D | Not |
| CSMD3 | ENST00000297405 | G/T | aCa/aAa | T604K | Not |
| KMT2D | ENST00000301067 | T/C | gAt/gGt | D5462G | Not |
| KMT2D | ENST00000301067 | G/A | Cga/Tga | R2099* | Not |
| KMT2D | ENST00000301067 | G/A | Cag/Tag | Q3265* | Not |
| KMT2D | ENST00000301067 | C/A | cGg/cTg | R755L | Not |
| KMT2D | ENST00000301067 | C/A | cGg/cTg | R1388L | Not |
| KMT2D | ENST00000301067 | G/A | gCc/gTc | A5212V | Not |
| KMT2D | ENST00000301067 | C/T | Ggg/Agg | G5295R | Not |
| KMT2D | ENST00000301067 | G/A | Cag/Tag | Q2004* | Not |
| KMT2D | ENST00000301067 | G/A | Caa/Taa | Q4322* | Not |
| NBPF1 | ENST00000430580 | T/A | aAg/aTg | K623M | Ongoing24 |
| NBPF1 | ENST00000430580 | T/C | aAa/aGa | K41R | Ongoing24 |
| NBPF1 | ENST00000430580 | C/A | Gaa/Taa | E688* | Ongoing24 |
| NBPF1 | ENST00000430580 | G/T | Ccc/Acc | P926T | Ongoing24 |
| RNF213 | ENST00000508628 | C/A | Ctc/Atc | L4751I | Ongoing24 |
| RNF213 | ENST00000508628 | G/C | Gaa/Caa | E4942Q | Ongoing24 |
| RNF213 | ENST00000508628 | C/T | gCc/gTc | A2744V | Ongoing24 |

*Stop codon gained;
**start codon lost.

PFS ongoing >24 months vs. not in DLBCL—In DLBCL, the most frequent gene mutations were RNF213, NBPF1, and BCL2 in patients who had PFS >24 months (3/7 [42.9%] each), and KMT2D (8/20 [40.0%]) and CSMD3 (8/20 [40.0%]) in patients who did not. Somatic mutation burden was lower in responders vs nonresponders, especially in germinal center B-cell-DLBCL, and in DLBCL pts with PFS >24 months vs not.

The above analysis identified gene variations among DLBCL, FL, and RT patients associated with response or durable PFS with a combination of ibrutinib and nivolumab. While ibrutinib inhibits Bruton's tyrosine kinase-dependent pathways, alternative gene pathway variants that may affect treatment outcomes were identified. Immune cell infiltration into the microenvironment relates to differential treatment response with this immune combination and is histology dependent.

Baseline TP53 Mutations and Molecular Remission are Prognostic Biomarkers of Benefit from Ibrutinib Treatment in Relapse/Refractory DLBCL

Baseline TP53 mutations and a 2-log₁₀ drop in ctDNA load after 2 courses of chemoimmunotherapy (molecular remission, MR) are both prognostic biomarkers in untreated diffuse large B-cell lymphoma (DLBCL). Their prognostic value in the setting of relapsed DLBCL treated with targeted agents is still poorly understood. The LYM1002 trial is a prospective phase 1/2a study aiming at testing the safety and activity of the combination of ibrutinib plus nivolumab in relapsed/refractory B-cell malignancies. Here, the prognostic impact of baseline mutations and MR in DLBCL treated with ibrutinib plus nivolumab within the LYM1002 trial was tested by using ctDNA.

### Methods

Inclusion criteria for this ancillary biological study was the availability of blood collected at baseline and C3D1. Where available, blood collected at the time of disease progression/end of therapy was also included in the analysis. CAPP-seq was used for ctDNA genotyping and ctDNA quantification. Assay sensitivity was 0.3%.

### Results

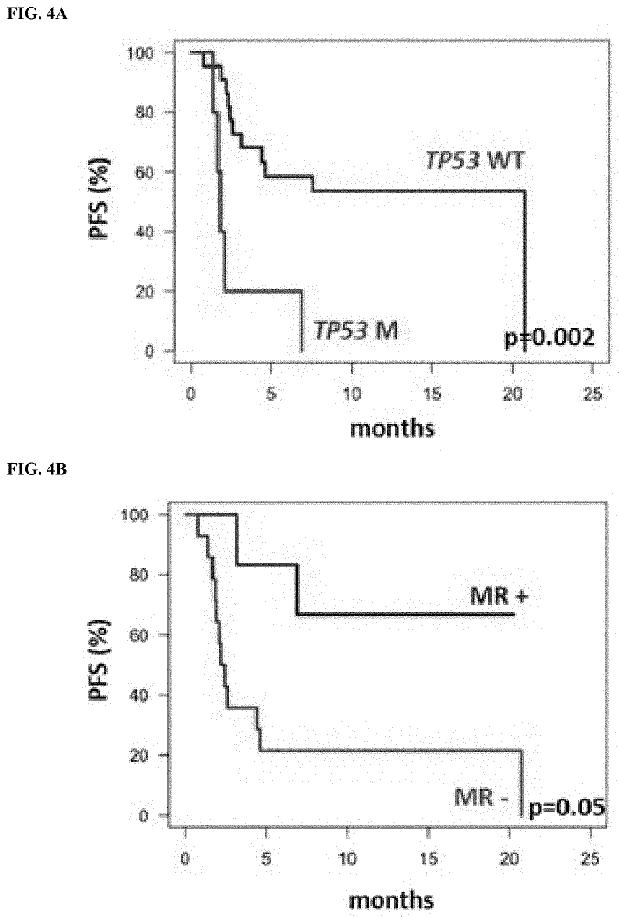

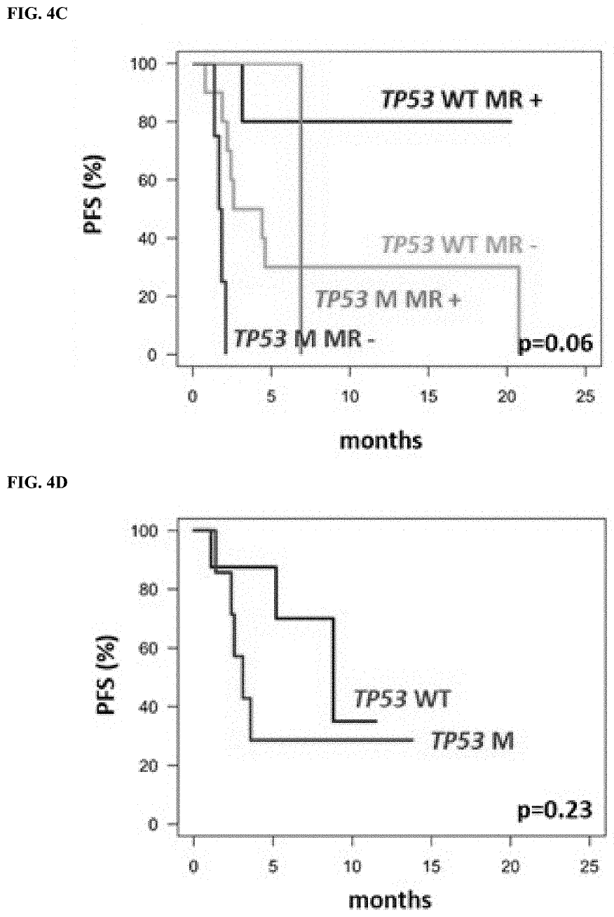

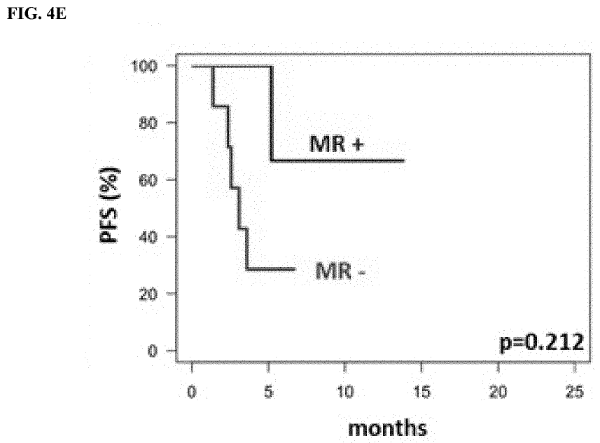

Among 37 relapsed/refractory DLBCL patients recruited in the LYM1002 trial, 27 fulfilled the inclusion criteria. Consistent with a relative enrichment of GCB DLBCL in the study cohort (GCB 78% vs ABC 5% vs intermediate 17%) genes recurrently affected by non-synonymous somatic mutations in >10% of patients included HIST1H1E, KMT2D, MEF2B TP53, BCL2, BTG1, EP300, ZNF292, MGA, HIST1H1C, XPO1, BTG1, CARD11, CREBBP, EZH2, PIM1, CIITA, DDX3X, MYC, TNFRSF14. After considering genes mutated in >10% of cases, only TP53 mutation status was significantly associated with inferior progression free survival (12-months PFS of 0% in TP53 mutated cases vs 12-months PFS of 53.6% in TP53 wild type cases; p=0.002) (FIG. 4A). A 2-login drop in ctDNA after 2 courses of ibrutinib plus nivolumab (MR) was associated with longer PFS (12-months PFS of 66.7% vs 21.4%; p=0.05) (FIG. 4B). A subgroup of relapsed/refractory DLBCL characterized by wild type TP53 at baseline and MR after 2 courses of ibrutinib plus nivolumab (19% of cases) showed promising long lasting remission (12-months PFS: 80%; p=0.06) (FIG. 4C). Among 10 patients provided with ctDNA collected at progression, a limited proportion (2 cases; 20%) acquired mutations in B-cell receptor signaling genes, including BTK and PLCG2 in one patient and in FOXO1 in the second patient. TP53 mutations observed in ctDNA samples from subjects with DLBCL are provided in Table 19.

Among 20 DLBCL transformed from chronic lymphocytic leukemia (CLL) (also known as Richter Syndrome) recruited in the LYM1002 trial, 14 fulfilled the inclusion criteria. Genes recurrently affected by non-synonymous somatic mutations in >10% of patients were TP53, NOTCH1, HIST1H1E, EGR2, SF3B1, ATM, ASXL1, CHEK2, MGA, NRAS. At variance with de novo DLBCL, baseline TP53 mutations did not significantly affect PFS in Richter Syndrome treated with ibrutinib plus nivolumab (FIG. 4D), which is consistent with the notion that ibrutinib overcomes, at least in part, the negative impact of TP53 abnormalities in CLL. In addition, consistent with the notion that ibrutinib does not eradicate minimal residual disease in CLL, only one Richter syndrome patient achieved MR after 2 courses of therapy (FIG. 4E).

### Conclusions

Baseline TP53 mutation status and MR after 2 courses are prognostic biomarkers of benefit from ibrutinib treatment in relapsed/refractory DLBCL but not in Richter Syndrome.

TABLE 19
TP53 mutations observed in ctDNA samples from subjects with DLBCL

| | | | | | | | | | | Variant |
|---|---|---|---|---|---|---|---|---|---|---|
| | | | | | | | | | | Allele |
| Sample | Cycle | Chr | Position | Ref | Var | Exon | Type | C. | P. | Frequency |
| ES10002004 | C1D1 | chr17 | 7577120 | C | T | EX8 | missense | c.818G > A | p.R273H | 12.37% |
| ES10002004 | C1D1 | chr17 | 7577556 | C | T | EX7 | missense | c.725G > A | p.C242Y | 12.24% |
| ES10002004 | C3D1 | chr17 | 7577120 | C | T | EX8 | missense | c.818G > A | p.R273H | 4.76% |
| ES10002004 | C3D1 | chr17 | 7577556 | C | T | EX7 | missense | c.725G > A | p.C242Y | 4.43% |
| ES10002004 | EOT | chr17 | 7577120 | C | T | EX8 | missense | c.818G > A | p.R273H | 7.15% |
| ES10002004 | EOT | chr17 | 7577556 | C | T | EX7 | missense | c.725G > A | p.C242Y | 4.61% |
| ES10003002 | C1D1 | chr17 | 7577539 | G | A | EX7 | missense | c.742C > T | p.R248W | 0.74% |
| ES10003002 | C1D1 | chr17 | 7577575 | A | G | EX7 | missense | c.706T > C | p.Y236H | 1.23% |
| ES10003002 | C1D1 | chr17 | 7577100 | T | C | EX8 | missense | c.838A > G | p.R280G | 2.11% |
| ES10003002 | C3D1 | chr17 | 7577539 | G | A | EX7 | missense | c.742C > T | p.R248W | 2.46% |
| ES10003002 | C3D1 | chr17 | 7577575 | A | G | EX7 | missense | c.706T > C | p.Y236H | 3.59% |
| ES10003002 | C3D1 | chr17 | 7577100 | T | C | EX8 | missense | c.838A > G | p.R280G | 1.62% |
| ES10003002 | EOT | chr17 | 7577100 | T | C | EX8 | missense | c.838A > G | p.R280G | 13.03% |
| ES10003002 | EOT | chr17 | 7577539 | G | A | EX7 | missense | c.742C > T | p.R248W | 4.05% |
| ES10003002 | EOT | chr17 | 7577575 | A | G | EX7 | missense | c.706T > C | p.Y236H | 2.96% |
| IL10001005 | C1D1 | chr17 | 7577498 | C | A | EX7 | splice-donor | c.782 + 1G > T | | 1.31% |
| IL10001005 | C3D1 | chr17 | 7577498 | C | A | EX7 | splice-donor | c.782 + 1G > T | | 4.96% |
| IL10001009 | C1D1 | chr17 | 7578406 | C | T | EX5 | missense | c.524G > A | p.R175H | 10.02% |
| IL10001009 | C3D1 | chr17 | 7578406 | C | T | EX5 | missense | c.524G > A | p.R175H | 3.73% |
| IL10001009 | EOT | chr17 | 7578406 | C | T | EX5 | missense | c.524G > A | p.R175H | 4.89% |
| IL10002008 | C1D1 | chr17 | 7577538 | C | A | EX7 | missense | c.743G > T | p.R248L | 28.13% |
| IL10002008 | C3D1 | chr17 | 7577538 | C | A | EX7 | missense | c.743G > T | p.R248L | 14.06% |
| IL10002008 | EOT | chr17 | 7577538 | C | A | EX7 | missense | c.743G > T | p.R248L | 6.30% |
| TR10001007 | C1D1 | chr17 | 7578551 | A | -GTACT | EX5 | frameshift | c.376-2_378Del5 | | 34.86% |
| TR10001007 | C3D1 | chr17 | 7578551 | A | -GTACT | EX5 | frameshift | c.376-2_378Del5 | | 2.27% |
| TR10001007 | EOT | chr17 | 7578551 | A | -GTACT | EX5 | frameshift | c.376-2_378Del5 | | 10.18% |
| US10001009 | C1D1 | chr17 | 7577093 | C | T | EX8 | missense | c.845G > A | p.R282Q | 47.37% |
| US10001009 | EOT | chr17 | 7577093 | C | T | EX8 | missense | c.845G > A | p.R282Q | 16.06% |

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated herein by reference, in its entirety.

### EMBODIMENTS

The following list of embodiments is intended to complement, rather than displace or supersede, the previous descriptions.

### Embodiment 1

A method of treating a B-cell malignancy in a subject, the method comprising:

administering to the subject a therapeutically effective amount of a combination of ibrutinib and an anti-PD-1 antibody to thereby treat the B-cell malignancy, wherein:
a) the B-cell malignancy is DLBCL and the subject has one or more mutations in genes selected from KLHL14, RNF213, CSMD3, BCL2, NBPF1, LRP1B, or a combination thereof, wherein the one or more mutations are listed in Table 4 or 6;
b) the B-cell malignancy is GCB-DLBCL and the subject has one or more mutations in genes selected from RNF213, NBPF1, or a combination thereof, wherein the one or more mutations are listed in Table 16;
c) the B-cell malignancy is FL and the subject has one or more mutations in genes selected from BCL2, CREBBP, KMT2D, MUC17, CIITA, FES, NCOA2, TPR, or a combination thereof, wherein the one or more mutations are listed in Table 8 or 10; or
d) the B-cell malignancy is RT and the subject has one or more mutations in genes selected from IRF2BP2, NBPF1, KLHL6, SETX, SF3B1, or a combination thereof, wherein the one or more mutations are listed in Table 12 or 14.

### Embodiment 2

The method of embodiment 1, wherein the B-cell malignancy is DLBCL and the subject has one or more mutations in genes selected from KLHL14, RNF213, CSMD3, BCL2, NBPF1, LRP1B, or a combination thereof, wherein the one or more mutations are listed in Table 4 or 6.

### Embodiment 3

The method of embodiment 2, wherein the subject has one or more mutations in KLHL14, RNF213, or a combination thereof, wherein the one or more mutations are listed in Table 4 or 6.

### Embodiment 4

The method of embodiment 1, wherein the B-cell malignancy is GCB-DLBCL and the subject has one or more mutations in genes selected from RNF213, NBPF1, or a combination thereof, wherein the one or more mutations are listed in Table 16.

### Embodiment 5

The method of embodiment 1, wherein the B-cell malignancy is FL and the subject has one or more mutations in genes selected from BCL2, CREBBP, KMT2D, MUC17, CITTA, FES, NCOA2, TPR, or a combination thereof, wherein the one or more mutations are listed in Table 8 or 10.

### Embodiment 6

The method of embodiment 5, wherein the subject has one or more mutations in BCL2, wherein the one or more mutations are listed in Table 8 or 10.

### Embodiment 7

The method of embodiment 1, wherein the B-cell malignancy is RT and the subject has one or more mutations in genes selected from IRF2BP2, NBPF1, KLHL6, SETX, SF3B1, or a combination thereof, wherein the one or more mutations are listed in Table 12 or 14.

### Embodiment 8

A method of treating a B-cell malignancy in a subject, the method comprising:

administering to the subject a therapeutically effective amount of a combination of ibrutinib and an anti-PD-1 antibody to thereby treat the B-cell malignancy, wherein:
a) the B-cell malignancy is DLBCL and the subject does not have one or more mutations in genes selected from TP53, EBF1, ADAMTS20, AKAP9, SOCS1, TNFRSF14, MYD88, NFKB1B, or a combination thereof, wherein the one or more mutations are listed in Table 4 or 6;
b) the B-cell malignancy is GCB-DLBCL and the subject does not have one or more mutations in genes selected from KMT2D, BCL2, CSMD3, CREBBP, EBF1, SGK1, or a combination thereof, wherein the one or more mutations are listed in Table 16;
c) the B-cell malignancy is FL and the subject does not have one or more mutations in genes selected from CREBBP, KMT2D, BCL2, STAT6, NBPF1, EZH2, or a combination thereof, wherein the one or more mutations are listed in Table 8 or 10; or
d) the B-cell malignancy is RT and the subject does not have one or more mutations in genes selected from ROS1, IGLL5, PASK, or a combination thereof, wherein the one or more mutations are listed in Table 12 or 14.

### Embodiment 9

The method of embodiment 8, wherein the B-cell malignancy is DLBCL and the subject does not have one or more mutations in genes selected from TP53, EBF1, ADAMTS20, AKAP9, SOCS1, TNFRSF14, MYD88, NFKB1B, or a combination thereof, wherein the one or more mutations are listed in Table 4 or 6.

### Embodiment 10

The method of embodiment 8, wherein the B-cell malignancy is GCB-DLBCL and the subject does not have one or more mutations in genes selected from KMT2D, BCL2, CSMD3, CREBBP, EBF1, SGK1, or a combination thereof, wherein the one or more mutations are listed in Table 16.

### Embodiment 11

The method of embodiment 8, wherein the B-cell malignancy is FL and the subject does not have one or more mutations in genes selected from CREBBP, KMT2D, BCL2, STAT6, NBPF1, EZH2, or a combination thereof, wherein the one or more mutations are listed in Table 8 or 10.

### Embodiment 12

The method of embodiment 8, wherein the B-cell malignancy is RT and the subject does not have one or more mutations in genes selected from ROS1, IGLL5, PASK, or a combination thereof, wherein the one or more mutations are listed in Table 12 or 14.

### Embodiment 13

The method of embodiment 12, wherein the subject does not have one or more mutations in ROS1, wherein the one or more mutations are listed in Table 12 or 14.

### Embodiment 14

The method of any one of the previous embodiments, wherein the therapeutically effective amount of the combination of ibrutinib and the anti-PD-1 antibody comprises 560 mg of the ibrutinib and 3 mg/kg of the anti-PD-1 antibody.

### Embodiment 15

The method of any one of the previous embodiments, wherein the anti-PD-1 antibody is administered intravenously and the ibrutinib is administered orally.

### Embodiment 16

The method of embodiment 15, wherein the anti-PD-1 antibody is administered on a 14-day cycle and the ibrutinib is administered once daily.

### Embodiment 17

The method of any one of the previous embodiments, wherein the anti-PD-1 antibody is nivolumab.

### Embodiment 18

The method of any one of the previous embodiments, wherein the treating results in a complete response (CR) or partial response (PR) in the subject.

### Embodiment 19

The method of any one of the previous embodiments, wherein the subject:
a) has DLBCL, FL, or RT (transformation from CLL/SLL only);
b) had ≥1 prior therapy (≥2 prior therapies for FL) but no more than 4 prior lines of treatment;
c) had an Eastern Cooperative Oncology Group (ECOG) performance status ≤2;
d) has measurable disease; and
e) has no prior ibrutinib or anti-PD-1 therapies.

### Embodiment 20

A method of predicting a likelihood of responsiveness to a combination of ibrutinib and an anti-PD-1 antibody in a subject having a B-cell malignancy, wherein:
a) the B-cell malignancy is DLBCL and the method comprises analyzing a sample from the subject for one or more mutations in genes selected from KLHL14, RNF213, CSMD3, BCL2, NBPF1, LRP1B, or a combination thereof, wherein the one or more mutations are listed in Table 4 or 6;
b) the B-cell malignancy is GCB-DLBCL and the method comprises analyzing a sample from the subject for one or more mutations in genes selected from RNF213, NBPF1, or a combination thereof, wherein the one or more mutations are listed in Table 16;
c) the B-cell malignancy is FL and the method comprises analyzing a sample from the subject for one or more mutations in genes selected from BCL2, CREBBP, KMT2D, MUC17, CIITA, FES, NCOA2, TPR, or a combination thereof, wherein the one or more mutations are listed in Table 8 or 10; or
d) the B-cell malignancy is RT and the method comprises analyzing a sample from the subject for one or more mutations in genes selected from IRF2BP2, NBPF1, KLHL6, SETX, SF3B1, or a combination thereof, wherein the one or more mutations are listed in Table 12 or 14;

wherein the one or more mutations in the genes are indicative of responsiveness to the combination.

### Embodiment 21

The method of embodiment 20, wherein the B-cell malignancy is DLBCL and the method comprises analyzing a sample from the subject for one or more mutations in genes selected from KLHL14, RNF213, CSMD3, BCL2, NBPF1, LRP1B, or a combination thereof, wherein the one or more mutations are listed in Table 4 or 6 and the one or more mutations in the genes are indicative of responsiveness to the combination.

### Embodiment 22

The method of embodiment 21, wherein the method comprises analyzing a sample from the subject for one or more mutations in genes selected from KLHL14, RNF213, or a combination thereof, wherein the one or more mutations are listed in Table 4 or 6 and the one or more mutations in the genes are indicative of responsiveness to the combination.

### Embodiment 23

The method of embodiment 20, wherein the B-cell malignancy is GCB-DLBCL and the method comprises analyzing a sample from the subject for one or more mutations in genes selected from RNF213, NBPF1, or a combination thereof, wherein the one or more mutations are listed in Table 16 and the one or more mutations in the genes are indicative of responsiveness to the combination.

### Embodiment 24

The method of embodiment 20, wherein the B-cell malignancy is FL and the method comprises analyzing a sample from the subject for one or more mutations in genes selected from BCL2, CREBBP, KMT2D, MUC17, CIITA, FES, NCOA2, TPR, or a combination thereof, wherein the one or more mutations are listed in Table 8 or 10 and the one or more mutations in the genes are indicative of responsiveness to the combination.

### Embodiment 25

The method of embodiment 24, wherein the method comprises analyzing a sample from the subject for one or more mutations in BCL2, wherein the one or more mutations are listed in Table 8 or 10 and the one or more mutations in the genes are indicative of responsiveness to the combination.

### Embodiment 26

The method of embodiment 20, wherein the B-cell malignancy is RT and the method comprises analyzing a sample from the subject for one or more mutations in genes selected from IRF2BP2, NBPF1, KLHL6, SETX, SF3B1, or a combination thereof, wherein the one or more mutations are listed in Table 12 or 14 and the one or more mutations in the genes are indicative of responsiveness to the combination.

### Embodiment 27

A method of predicting a likelihood of nonresponsiveness to a combination of ibrutinib and an anti-PD-1 antibody in a subject having a B-cell malignancy, wherein:
a) the B-cell malignancy is DLBCL and the method comprises analyzing a sample from the subject for one or more mutations in genes selected from TP53, EBF1, ADAMTS20, AKAP9, SOCS1, TNFRSF14, MYD88, NFKB1B, or a combination thereof, wherein the one or more mutations are listed in Table 4 or 6;
b) the B-cell malignancy is GCB-DLBCL and the method comprises analyzing a sample from the subject for one or more mutations in genes selected from KMT2D, BCL2, CSMD3, CREBBP, EBF1, SGK1, or a combination thereof, wherein the one or more mutations are listed in Table 16;
c) the B-cell malignancy is FL and the method comprises analyzing a sample from the subject for one or more mutations in genes selected from CREBBP, KMT2D, BCL2, STAT6, NBPF1, EZH2, or a combination thereof, wherein the one or more mutations are listed in Table 8 or 10; or
d) the B-cell malignancy is RT and the method comprises analyzing a sample from the subject for one or more mutations in genes selected from ROS1, IGLL5, PASK, or a combination thereof, wherein the one or more mutations are listed in Table 12 or 14;

wherein the one or more mutations in the genes is indicative of nonresponsiveness to the combination.

### Embodiment 28

The method of embodiment 27, wherein the B-cell malignancy is DLBCL and the method comprises analyzing a sample from the subject for one or more mutations in genes selected from TP53, EBF1, ADAMTS20, AKAP9, SOCS1, TNFRSF14, MYD88, NFKB1B, or a combination thereof, wherein the one or more mutations are listed in Table 4 or 6 and the one or more mutations in the genes is indicative of nonresponsiveness to the combination.

### Embodiment 29

The method of embodiment 27, wherein the B-cell malignancy is GCB-DLBCL and the method comprises analyzing a sample from the subject for one or more mutations in genes selected from KMT2D, BCL2, CSMD3, CREBBP, EBF1, SGK1, or a combination thereof, wherein the one or more mutations are listed in Table 16 and the one or more mutations in the genes is indicative of nonresponsiveness to the combination.

### Embodiment 30

The method of embodiment 27, wherein the B-cell malignancy is FL and the method comprises analyzing a sample from the subject for one or more mutations in genes selected from CREBBP, KMT2D, BCL2, STAT6, NBPF1, EZH2, or a combination thereof, wherein the one or more mutations are listed in Table 8 or 10 and the one or more mutations in the genes is indicative of nonresponsiveness to the combination.

### Embodiment 31

The method of embodiment 27, wherein the B-cell malignancy is RT and the method comprises analyzing a sample from the subject for one or more mutations in genes selected from ROS1, IGLL5, PASK, or a combination thereof, wherein the one or more mutations are listed in Table 12 or 14 and the one or more mutations in the genes is indicative of nonresponsiveness to the combination.

### Embodiment 32

The method of embodiment 31, wherein the method comprises analyzing a sample from the subject for one or more mutations in ROS1, wherein the one or more mutations are listed in Table 12 or 14 and the one or more mutations are indicative of nonresponsiveness to the combination.

### Embodiment 33

The method of any one of embodiments 20-32, wherein the subject:
a) has DLBCL, FL, or RT (transformation from CLL/SLL only);
b) had ≥1 prior therapy (≥2 prior therapies for FL) but no more than 4 prior lines of treatment;
c) had an Eastern Cooperative Oncology Group (ECOG) performance status ≤2;
d) has measurable disease; and
e) has no prior ibrutinib or anti-PD-1 therapies.

### Embodiment 34

The method of any one of embodiments 20-33, further comprising administering a therapeutically effective amount of the combination of ibrutinib and an anti-PD-1 antibody to the subject to thereby treat the B-cell malignancy if the subject has the one or more mutations in genes that are indicative of responsiveness to the combination and/or a lack of the one or more mutations in genes that are indicative of nonresponsiveness to the combination.

### Embodiment 35

The method of embodiment 34, wherein the therapeutically effective amount of the combination of ibrutinib and the anti-PD-1 antibody comprises 560 mg of the ibrutinib and 3 mg/kg of the anti-PD-1 antibody.

### Embodiment 36

The method of embodiment 34 or 35, wherein the anti-PD-1 antibody is administered intravenously and the ibrutinib is administered orally.

### Embodiment 37

The method of embodiment 36, wherein the anti-PD-1 antibody is administered on a 14-day cycle and the ibrutinib is administered once daily.

### Embodiment 38

The method of any one of the embodiments 34-37, wherein the anti-PD-1 antibody is nivolumab.

### Embodiment 39

The method of any one of embodiments 34-38, wherein the treating results in a complete response (CR) or partial response (PR) in the subject.

What is claimed:

1. A method of treating a B-cell malignancy in a subject, the method comprising:
administering to the subject a therapeutically effective amount of a combination of ibrutinib and an anti-PD-1 antibody to thereby treat the B-cell malignancy, wherein:
a) the B-cell malignancy is DLBCL and the subject has one or more mutations in genes selected from KLHL14, RNF213, CSMD3, BCL2, NBPF1, LRP1B, or a combination thereof, wherein the one or more mutations are listed in Table 4 or 6;
b) the B-cell malignancy is GCB-DLBCL and the subject has one or more mutations in genes selected from RNF213, NBPF1, or a combination thereof, wherein the one or more mutations are listed in Table 16;
c) the B-cell malignancy is FL and the subject has one or more mutations in genes selected from BCL2, CREBBP, KMT2D, MUC17, CITTA, FES, NCOA2, TPR, or a combination thereof, wherein the one or more mutations are listed in Table 8 or 10; or
d) the B-cell malignancy is RT and the subject has one or more mutations in genes selected from IRF2BP2, NBPF1, KLHL6, SETX, SF3B1, or a combination thereof, wherein the one or more mutations are listed in Table 12 or 14.

2. The method of claim 1, wherein the B-cell malignancy is DLBCL and the subject has one or more mutations in genes selected from KLHL14, RNF213, CSMD3, BCL2, NBPF1, LRP1B, or a combination thereof, wherein the one or more mutations are listed in Table 4 or 6.

3. The method of claim 2, wherein the subject has one or more mutations in KLHL14, RNF213, or a combination thereof, wherein the one or more mutations are listed in Table 4 or 6.

4. The method of claim 1, wherein the B-cell malignancy is GCB-DLBCL and the subject has one or more mutations in genes selected from RNF213, NBPF1, or a combination thereof, wherein the one or more mutations are listed in Table 16.

5. The method of claim 1, wherein the B-cell malignancy is FL and the subject has one or more mutations in genes selected from BCL2, CREBBP, KMT2D, MUC17, CITTA, FES, NCOA2, TPR, or a combination thereof, wherein the one or more mutations are listed in Table 8 or 10.

6. The method of claim 5, wherein the subject has one or more mutations in BCL2, wherein the one or more mutations are listed in Table 8 or 10.

7. The method of claim 1, wherein the B-cell malignancy is RT and the subject has one or more mutations in genes selected from IRF2BP2, NBPF1, KLHL6, SETX, SF3B1, or a combination thereof, wherein the one or more mutations are listed in Table 12 or 14.

8. The method of claim 1, comprising, prior to the administering:
a) analyzing a sample from a subject having DLBCL for one or more mutations in genes selected from KLHL14, RNF213, CSMD3, BCL2, NBPF1, LRP1B, or a combination thereof, wherein the one or more mutations are listed in Table 4 or 6;
b) analyzing a sample from a subject having GCB-DLBCL for one or more mutations in genes selected from RNF213, NBPF1, or a combination thereof, wherein the one or more mutations are listed in Table 16;
c) analyzing a sample from a subject having FL for one or more mutations in genes selected from BCL2, CREBBP, KMT2D, MUC17, CITTA, FES, NCOA2, TPR, or a combination thereof, wherein the one or more mutations are listed in Table 8 or 10; or
d) analyzing a sample from a subject having RT for one or more mutations in genes selected from IRF2BP2, NBPF1, KLHL6, SETX, SF3B1, or a combination thereof, wherein the one or more mutations are listed in Table 12 or 14;
wherein the one or more mutations in the genes are indicative of responsiveness to the combination.

9. The method of claim 1, wherein the therapeutically effective amount of the combination of ibrutinib and the anti-PD-1 antibody comprises 560 mg of the ibrutinib and 3 mg/kg of the anti-PD-1 antibody.

10. The method of claim 1, wherein the anti-PD-1 antibody is administered intravenously and the ibrutinib is administered orally.

11. The method of claim 10, wherein the anti-PD-1 antibody is administered on a 14-day cycle and the ibrutinib is administered once daily.

12. The method of claim 1, wherein the anti-PD-1 antibody is nivolumab.

13. The method of claim 1, wherein the treating results in a complete response (CR) or partial response (PR) in the subject.

14. The method of claim 1, wherein the subject:
a) has DLBCL, FL, or RT (transformation from CLL/SLL only);
b) had ≥1 prior therapy (≥2 prior therapies for FL) but no more than 4 prior lines of treatment;
c) had an Eastern Cooperative Oncology Group (ECOG) performance status ≤2;
d) has measurable disease; and
e) has no prior ibrutinib or anti-PD-1 therapies.

15. A method of treating a B-cell malignancy in a subject, the method comprising:
administering to the subject a therapeutically effective amount of a combination of ibrutinib and an anti-PD-1 antibody to thereby treat the B-cell malignancy, wherein:
a) the B-cell malignancy is DLBCL and the subject does not have one or more mutations in genes selected from TP53, EBF1, ADAMTS20, AKAP9, SOCS1, TNFRSF14, MYD88, NFKB1B, or a combination thereof, wherein the one or more mutations are listed in Table 4 or 6;
b) the B-cell malignancy is GCB-DLBCL and the subject does not have one or more mutations in genes selected from KMT2D, BCL2, CSMD3, CREBBP, EBF1, SGK1, or a combination thereof, wherein the one or more mutations are listed in Table 16;
c) the B-cell malignancy is FL and the subject does not have one or more mutations in genes selected from CREBBP, KMT2D, BCL2, STATE, NBPF1, EZH2, or a combination thereof, wherein the one or more mutations are listed in Table 8 or 10; or
d) the B-cell malignancy is RT and the subject does not have one or more mutations in genes selected from ROS1, IGLL5, PASK, or a combination thereof, wherein the one or more mutations are listed in Table 12 or 14.

16. The method of claim 15, wherein the B-cell malignancy is DLBCL and the subject does not have one or more mutations in genes selected from TP53, EBF1, ADAMTS20, AKAP9, SOCS1, TNFRSF14, MYD88, NFKB1B, or a combination thereof, wherein the one or more mutations are listed in Table 4 or 6.

17. The method of claim 15, wherein the B-cell malignancy is GCB-DLBCL and the subject does not have one or more mutations in genes selected from KMT2D, BCL2, CSMD3, CREBBP, EBF1, SGK1, or a combination thereof, wherein the one or more mutations are listed in Table 16.

18. The method of claim 15, wherein the B-cell malignancy is FL and the subject does not have one or more mutations in genes selected from CREBBP, KMT2D, BCL2, STATE, NBPF1, EZH2, or a combination thereof, wherein the one or more mutations are listed in Table 8 or 10.

19. The method of claim 15, wherein the B-cell malignancy is RT and the subject does not have one or more mutations in genes selected from ROS1, IGLL5, PASK, or a combination thereof, wherein the one or more mutations are listed in Table 12 or 14.

20. The method of claim 19, wherein the subject does not have one or more mutations in ROS1, wherein the one or more mutations are listed in Table 12 or 14.

21. The method of claim 15, comprising, prior to the administering:
a) analyzing a sample from a subject having DLBCL for one or more mutations in genes selected from TP53, EBF1, ADAMTS20, AKAP9, SOCS1, TNFRSF14, MYD88, NFKB1B, or a combination thereof, wherein the one or more mutations are listed in Table 4 or 6;
b) analyzing a sample from a subject having GCB-DLBCL for one or more mutations in genes selected from KMT2D, BCL2, CSMD3, CREBBP, EBF1, SGK1, or a combination thereof, wherein the one or more mutations are listed in Table 16;
c) analyzing a sample from a subject having FL for one or more mutations in genes selected from CREBBP, KMT2D, BCL2, STATE, NBPF1, EZH2, or a combination thereof, wherein the one or more mutations are listed in Table 8 or 10; or
d) analyzing a sample from a subject having RT for one or more mutations in genes selected from ROS1, IGLL5, PASK, or a combination thereof, wherein the one or more mutations are listed in Table 12 or 14;
wherein the one or more mutations in the genes is indicative of nonresponsiveness to the combination.

22. The method of claim 15, wherein the therapeutically effective amount of the combination of ibrutinib and the anti-PD-1 antibody comprises 560 mg of the ibrutinib and 3 mg/kg of the anti-PD-1 antibody.

23. The method of claim 15, wherein the anti-PD-1 antibody is administered intravenously and the ibrutinib is administered orally.

24. The method of claim 23, wherein the anti-PD-1 antibody is administered on a 14-day cycle and the ibrutinib is administered once daily.

25. The method of claim 15, wherein the anti-PD-1 antibody is nivolumab.

26. The method of claim 15, wherein the treating results in a complete response (CR) or partial response (PR) in the subject.

27. The method of claim 15, wherein the subject:
a) has DLBCL, FL, or RT (transformation from CLL/SLL only);
b) had ≥1 prior therapy (≥2 prior therapies for FL) but no more than 4 prior lines of treatment;
c) had an Eastern Cooperative Oncology Group (ECOG) performance status ≤2;
d) has measurable disease; and
e) has no prior ibrutinib or anti-PD-1 therapies.
